(19) United States
(12) Patent Application Publication
(10) Pub. No.: US 2003/0113714 A1
(43) Pub. Date: Jun. 19, 2003

(54) Biological control of nanoparticles

(76) Inventors: Angela M. Belcher, Lexington, MA (US); Richard E. Smalley, Houston, TX (US); Esther Ryan, San Jose, CA (US); Seung-Wuk Lee, Austin, TX (US)

Correspondence Address: Sanford E. Warren, Jr., Gardere Wynne Sewell LLP, 3000 Thanksgiving Tower, 1601 Elm Street, Suite 3000, Dallas, TX, 75201-4767, US

(21) Appl. No.: 10/254,446

(22) Filed: Sep. 25, 2002

Related U.S. Application Data
(60) Provisional application No. 60/325,664, filed on Sep. 28, 2001.

(51) Int. Cl. H01L 21/00; C12Q 1/70; C12Q 1/68; G01N 33/554; G01N 33/569

(52) U.S. Cl. 435/5; 435/6; 435/7.32; 438/1

(57) ABSTRACT

The present invention includes compositions and methods for selective binding of amino acid oligomers to semiconductor and elemental carbon-containing materials. One form of the present invention is a method for controlling the particle size of the semiconductor or elemental carbon-containing material by interacting an amino acid oligomer that specifically binds the material with solutions that can result in the formation of the material. The same method can be used to control the aspect ratio of the nanocrystal particles of the semiconductor material. Another form of the present invention is a method to create nanowires from the semiconductor or elemental carbon-containing material. Yet another form of the present invention is a biologic scaffold comprising a substrate capable of binding one or more biologic materials, one or more biologic materials attached to the substrate, and one or more elemental carbon-containing molecules attached to one or more biologic materials.

This application claims priority from Provisional Patent Application Serial No. 60/325,664, filed on Sep. 28, 2001.

The research carried out in the subject application was supported in part by grants from the Army Research Office (DADD19-99-0155).

### FIELD OF THE INVENTION

The present invention is directed to the selective recognition of various materials in general and, specifically, toward surface recognition of semiconductor materials and elemental carbon-containing materials using organic polymers.

### BACKGROUND OF THE INVENTION

In biologic systems, organic molecules exhibit a remarkable level of control over the nucleation and mineral phase of inorganic materials such as calcium carbonate and silica, and over the assembly of crystallites and other nanoscale building blocks into complex structures required for biologic function. This control could, in theory, be applied to materials with interesting magnetic, electrical or optical properties.

Materials produced by biologic processes are typically soft, and consist of a surprisingly simple collection of molecular building blocks (i.e., lipids, peptides, and nucleic acids) arranged in astoundingly complex architectures. Unlike the semiconductor industry, which relies on a serial lithographic processing approach for constructing the smallest features on an integrated circuit, living organisms execute their architectural “blueprints” using both covalent and non-covalent forces acting simultaneously upon many molecular components. Furthermore, these structures can often elegantly rearrange between two or more usable forms without changing any of the molecular constituents.

The use of “biologic” materials to process the next generation of microelectronic, optic and magnetic devices provides a possible solution to resolving the limitations of traditional processing methods. The critical factors in this approach are identifying the appropriate compatibilities and combinations of biologic-inorganic-organic materials, the synthetic process and recognition for creating unique and specific combinations, and the understanding the synthesis of the appropriate building blocks.

### SUMMARY OF THE INVENTION

The present invention is based on the selection, production, isolation and characterization of organic polymers, e.g., peptides, with enhanced selectivity to various organic and inorganic materials. In one embodiment of the present invention, biologic materials, e.g., combinatorial libraries such as a phage display library, cause directed molecular recognition of a target taking advantage of iterative rounds of peptide evolution. Organic polymers (e.g., peptides) may be created and derived that attach with high specificity to a wide range of materials including but not limited to semiconductor surfaces and elemental carbon-containing compounds such as carbon nanotubes and graphite. Furthermore, the invention allows for the selective isolation of organic recognition molecules (e.g., organic polymers) that may specifically recognize a specific orientation, shape or structure of the biologic material (e.g., crystallographic shape or orientation), whether or not a composition of the structurally similar material is used.

In one embodiment of the present invention, a biologic scaffold is disclosed. The scaffold includes a substrate capable of binding one or more biologic materials, one or more biologic materials attached to the substrate, and one or more elemental carbon-containing molecules attached to the biologic materials. In another embodiment of the present invention, a biologic scaffold is disclosed that includes a substrate capable of binding one or more biologic materials, a first biologic material attached to the substrate and a second biologic material attached to the first biologic material, and one or more elemental carbon-containing molecules attached to the second biologic material.

In another embodiment of the present invention, the biologic scaffold includes a substrate capable of binding one or more bacteriophages, one or more bacteriophages attached to the substrate, one or more peptides that recognize a portion of the bacteriophage, and one or more elemental carbon-containing molecules that recognize the peptide.

In another embodiment of the present invention, a method of making a biologic scaffold is disclosed. The method includes providing a substrate capable of binding one or more biologic materials, attaching one or more biologic materials to the substrate, and contacting one or more elemental carbon-containing molecules with the biologic material to form a biologic scaffold.

In another embodiment of the present invention, a molecule is described. The molecule contains an organic polymer that selectively recognizes an elemental carbon-containing molecule.

In another embodiment of the present invention, a method for directed semiconductor formation is described. The method includes the steps of contacting a molecule that binds a predetermined face specificity semiconductor material with a first ion to create a semiconductor material precursor and adding a second ion to the semiconductor material precursor, wherein the molecule directs formation of the predetermined face specific semiconductor material. The molecule may include an amino acid oligomer or peptide, which may be on the surface of a bacteriophage as part of, e.g., a chimeric coat protein. The molecule may even be a nucleic acid oligomer and may be selected from a combinatorial library. The molecule may be an amino acid polymer of between about 7 and 20 amino acids. The present invention also encompasses a semiconductor material made using the method of the present invention.

Uses for the controlled crystals directed and grown using the materials and methods of the present invention include materials with novel optical, electronic and magnetic properties. As will be known to those of skill in the art, the detailed optical, electronic and magnetic properties may be directed by the formation of semiconductor crystal by, e.g., patterning the devices, which using the present invention may include layering or laying down patterns to create crystal formation in patterns, layers or even both.

Another use of the patterns and/or layers formed using the present invention is the formation of semiconductor devices for high density magnetic storage. Another design may be for the formation of transistors for use in, e.g., quantum computing. Yet another use for the patterns, designs and novel materials made with the present invention include imaging and imaging contrast agent for medical applications.

One such use for the directed formation of semiconductors and semiconductor crystals and designs include information storage based on quantum dot patterns, e.g., identification of friend or foe in military or even personnel situations. The quantum dots could be used to identify individual soldiers or personnel using identification in fabric, in armor or on the person. Alternatively, the dots may be used in coding the fabric of money. Yet another use for the present invention is to create bi and multi-functional peptides for drug delivery in trapping the drug to be delivered using the peptides of the present invention. Yet another use is for in vivo and vitro diagnostics based on gene or protein expression by drug trapping using the peptides to deliver a drug.

For a more complete understanding of the features and advantages of the present invention, reference is now made to the detailed description of the invention along with the accompanying figures.

### BRIEF DESCRIPTION OF THE FIGURES

For more complete understanding of the features and advantages of the present invention, reference is now made to the detailed description of the invention along with the accompanying FIGURES.

FIG. 1 depicts selected random amino acid sequences in accordance with the present invention;

FIG. 2 depicts XPS spectra of structures in accordance with the present invention;

FIG. 3 depicts phage recognition of heterostructures in accordance with the present invention;

FIGS. 4-8 depict specific amino acid sequences in accordance with the present invention;

FIG. 9 depicts the peptide insert structure of the phage libraries in accordance with the present invention;

FIG. 10 depicts the various amino acid substitutions in the third and fourth rounds of selection in accordance with the present invention;

FIG. 11 depicts the amino acid substitutions after the fifth round of selection in accordance with the present invention;

FIG. 12 depicts the nanowire made from the ZnS nanoparticles in accordance with the present invention;

FIG. 13 depicts organic polymer (e.g., peptide) sequences obtained from PhD-C7C library selection against carbon planchet in accordance with the present invention;

FIG. 14 depicts organic polymer (e.g., peptide) sequences obtained from PhD-12 library selection against carbon planchet in accordance with the present invention;

FIG. 15 depicts organic polymer (e.g., peptide) sequences obtained from pHD-12 library selection against SWNT paste aggregates in accordance with the present invention;

FIG. 16 depicts organic polymer (e.g., peptide) sequences obtained from PhD-12 library selection against HOPG in accordance with the present invention;

FIG. 17 depicts binding efficiencies of various phage clones to SWNT paste aggregates in accordance with the present invention;

FIG. 18 depicts binding efficiencies of various phage clones to carbon planchet in accordance with the present invention;

FIG. 19 depicts confocal images of various phage clones bound to carbon planchet in accordance with the present invention;

FIG. 20 depicts confocal images of various biotinylated peptides bound to carbon planchet in accordance with the present invention;

FIG. 21 depicts confocal images of various phage clones bound to wet SWNT paste in accordance with the present invention;

FIG. 22 depicts AFM images of phage clones on HOPG in accordance with the present invention;

FIG. 23 depicts a schematic diagram of an SWNT purifying negative column;

FIG. 24 depicts a schematic diagram of phage binding to SWNT (phage-SWNT);

FIG. 25 depicts a schematic diagram of n-type SWNT modification using SWNT binding peptides;

FIG. 26 depicts a schematic diagram for the application of SWNT as a drug releasing system; and

FIG. 27 depicts a schematic diagram for the application of SWNTs in cancer medication.

### DETAILED DESCRIPTION OF THE INVENTION

Although making and using various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention, and do not delimit the scope of the invention.

Terms used herein have meanings as commonly understood by a person of ordinary skill in the areas relevant to the present invention. Terms such as “a,” “an,” and “the” are not intended to refer to only a singular entity, but include the general class of which a specific example may be used for illustration. The terminology herein is used to describe specific embodiments of the invention, but their usage does not limit the invention, except as outlined in the claims.

The terminology herein is used to describe specific embodiments of the invention, but their usage does not limit the invention, except as outlined in the claims. As used throughout the present specification, the terms “quantum dots”, “nanoparticles”, and “particles” are used interchangeably.

As used herein, the term “biologic material” and/or “biologic material” refers to a virus, bacteriophage, bacteria, peptide, protein, amino acid, steroid, drug, chromophore, antibody, enzyme, single-stranded or double-stranded nucleic acid, and any chemical modifications thereof. The biologic material may self-assemble to form a dry thin film on the contacting surface of a substrate. Self-assembly may permit random or uniform alignment of the biologic material on the surface. In addition, the biologic material may form a dry thin film that is externally controlled by solvent concentration, application of an electric and or magnetic field, optics, or other chemical or field interactions. As used herein, biologic material and “organic polymer” and “polymeric organic material” may be used interchangeably. As used herein, organic polymer refers to multiple units of organic material, wherein the organic material includes several “monomers” that may be the same or different. For example, proteins, antibodies, peptides, nucleic acids, chimeric molecules, drugs, and other carbon-containing materials known to exist in biologic systems (e.g., eukaryotic organisms) are illustrations of organic polymers. Other organic polymers may be derivatives or analogs of biologic polymers that contain one or more biologic monomers in combinations with synthetic monomers that may mimic those found naturally.

The term “inorganic molecule” or “inorganic compound” refers to compounds such as, e.g., indium tin oxide, doping agents, metals, minerals, radioisotope, salt, and combinations, thereof. Metals may include Ba, Sr, Ti, Bi, Ta, Zr, Fe, Ni, Mn, Pb, La, Li, Na, K, Rb, Cs, Fr, Be, Mg, Ca, Nb, Tl, Hg, Cu, Co, Rh, Sc, or Y. Inorganic compounds may include, e.g., high dielectric constant materials (insulators) such as barium strontium titanate, barium zirconate titanate, lead zirconate titanate, lead lanthanum titanate, strontium titanate, barium titanate, barium magnesium fluoride, bismuth titanate, strontium bismuth tantalite, and strontium bismuth tantalite niobate, or variations, thereof, known to those of ordinary skill in the art.

The term “organic molecule” or “organic compound” refers to compounds containing carbon alone or in combination, such as nucleotides, polynucleotides, nucleosides, steroids, DNA, RNA, peptides, protein, antibodies, enzymes, carbohydrate, lipids, conducting polymers, drugs, and combinations, thereof. A drug may include an antibiotic, antimicrobial, anti-inflammatory, analgesic, antihistamine, and any agent used therapeutically or prophylactically against mammalian pathologic (or potentially pathologic) conditions.

The term “elemental carbon-containing molecule” generally refers to allotropic forms of carbon. Examples include, but are not limited to, diamond, graphite, activated carbon, carbon₆₀, carbon black, industrial carbon, charcoal, coke, and steel. Other examples include, but are not limited to carbon planchet, highly ordered pyrolytic graphite (HOPG), single-walled nanotube (SWNT), single-walled nanotube paste, multi-walled nanotube, multi-walled nanotube paste as well as metal impregnated carbon-containing materials.

As used herein, a “substrate” may be a microfabricated solid surface to which molecules attach through either covalent or non-covalent bonds and includes, e.g., silicon, Langmuir-Bodgett films, functionalized glass, germanium, ceramic, silicon, a semiconductor material, PTFE, carbon, polycarbonate, mica, mylar, plastic, quartz, polystyrene, gallium arsenide, gold, silver, metal, metal alloy, fabric, and combinations thereof capable of having functional groups such as amino, carboxyl, thiol or hydroxyl incorporated on its surface. Similarly, the substrate may be an organic material such as a protein, mammalian cell, antibody, organ, or tissue with a surface to which a biologic material may attach. The surface may be large or small and not necessarily uniform but should act as a contacting surface (not necessarily in monolayer). The substrate may be porous, planar or nonplanar. The substrate includes a contacting surface that may be the substrate itself or a second layer (e.g., substrate or biologic material with a contacting surface) made of organic or inorganic molecules and to which organic or inorganic molecules may contact.

The inventors have previously shown that peptides may bind to semiconductor material. Semiconductor materials useful in binding peptides include, but are not limited to gallium arsenide, indium phosphate, gallium nitrate, zinc sulfide, aluminum arsenide, aluminum gallium arsenide, cadmium sulfide, cadmium selenide, zinc selenide, lead sulfide, boron nitride and silicon.

Semiconductor nanocrystals exhibit size and shape-dependent optical and electrical properties. These diverse properties result in their potential applications in a variety of devices such as light emitting diodes (LED), single electron transistors, photovoltaics, optical and magnetic memories, and diagnostic markers and sensors. Control of particle size, shape and phase is also critical in protective coatings such as car paint and in pigments such as house paints. The semiconductor materials may be engineered to be of certain shapes and sizes, wherein the optical and electrical properties of these semiconductor materials may best be exploited for use in numerous devices.

The present inventors have further developed a means of nucleating nanoparticles and directing their self-assembly. The main features of the peptides are their ability to recognize and bind technologically important materials with face specificity, to nucleate size-constrained crystalline semiconductor materials, and to control the crystallographic phase of nucleated nanoparticles. The peptides can also control the aspect ratio of the materials and therefore, the optical properties.

Briefly, the facility with which biologic systems assemble immensely complicated structure on an exceedingly minute scale has motivated a great deal of interest in the desire to identify non-biologic systems that can behave in a similar fashion. Of particular value would be methods that could be applied to materials with interesting electronic or optical properties, but natural evolution has not selected for interactions between biomolecules and such materials.

The present invention is based on recognition that biologic systems efficiently and accurately assemble nanoscale building blocks into complex and functionally sophisticated structures with high perfection, controlled size and compositional uniformity.

One method of providing a random organic polymer pool is using a Phage-display library. A Phage-display library is a combinatorial library of random peptides containing between 7 and 12 amino acids fused to the pIII coat protein of M13 coliphage, providing different peptides that are reactive with crystalline semiconductor structures or other materials. Five copies of the pIII coat protein are located on one end of the phage particle, accounting for 10-16 nm of the particle. The phage-display approach provides a physical linkage between the peptide substrate interaction and the DNA that encodes that interaction.

Peptide sequences have been developed with affinities for various materials such as semiconductors, and elemental carbon-containing molecules such as carbon nanotubes and graphite. Five different single-crystal semiconductors, GaAs (100), GaAs (111)A, GaAs(111)B, InP(100) and Si(100), were used in the following examples. These semiconductors allowed for systematic evaluation of the peptide interactions and confirmation of the general utility of the methodology of the present invention for different crystalline structures. In addition, elemental carbon-containing molecules such as carbon planchets, highly ordered pyrolytic graphite (HOPG), and single-walled nanotube (SWNT) paste were used.

Using a Phage-display library, protein sequences that successfully bound to the specific crystal were eluted from the surface, amplified by, e.g., a million-fold, and reacted against the substrate under more stringent conditions. This procedure was repeated between three and seven times to select the phage in the library with the most specific binding peptides. After, e.g., the third, fourth and fifth rounds of phage selection, crystal-specific phage were isolated and their DNA sequenced, identifying the peptide binding that is selective for the crystal composition (for example, binding to GaAs but not to Si) and crystalline face (for example, binding to (100) GaAs, but not to (111)B GaAs).

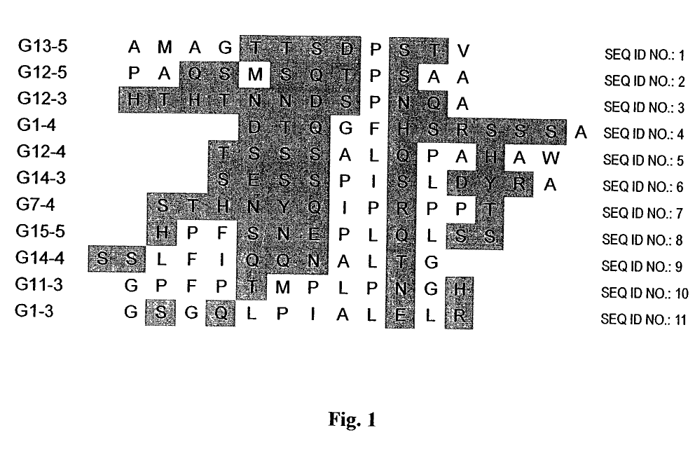

Twenty clones selected from GaAs(100) were analyzed to determine epitope binding domains by amino-acid functionality analysis to the GaAs surface. The partial peptide sequences of the modified pIII or pVIII protein are shown in FIG. 1, revealing similar binding domains among peptides exposed to GaAs. With increasing number of exposures to a GaAs surface, the number of uncharged polar and Lewis-base functional groups increased. Phage clones from third, fourth and fifth round sequencing contained on average 30%, 40% and 44% polar functional groups, respectively, while the fraction of Lewis-base functional groups increased at the same time from 41% to 48% to 55%. The observed increase in Lewis bases, which should constitute only 34% of the functional groups in random 12-mer peptides from our library, suggests that interactions between Lewis bases on the peptides and Lewis-acid sites on the GaAs surface may mediate the selective binding exhibited by these peptides.

The expected structure of the modified 12-mers selected from the library may be an extended conformation, which seems likely for small peptides, making the peptide much longer than the unit cell (5.65 A°) of GaAs. Therefore, only small binding domains would be necessary for the peptide to recognize a GaAs crystal. These short peptide domains, highlighted in FIG. 1, contain serine- and threonine-rich regions in addition to the presence of amine Lewis bases, such as asparagine and glutamine. To determine the exact binding sequence, the surfaces have been screened with shorter libraries, including 7-mer and disulphide constrained 7-mer libraries. Using these shorter libraries that reduce the size and flexibility of the binding domain, fewer peptide-surface interactions are allowed, yielding the expected increase in the strength of interactions between generations of selection.

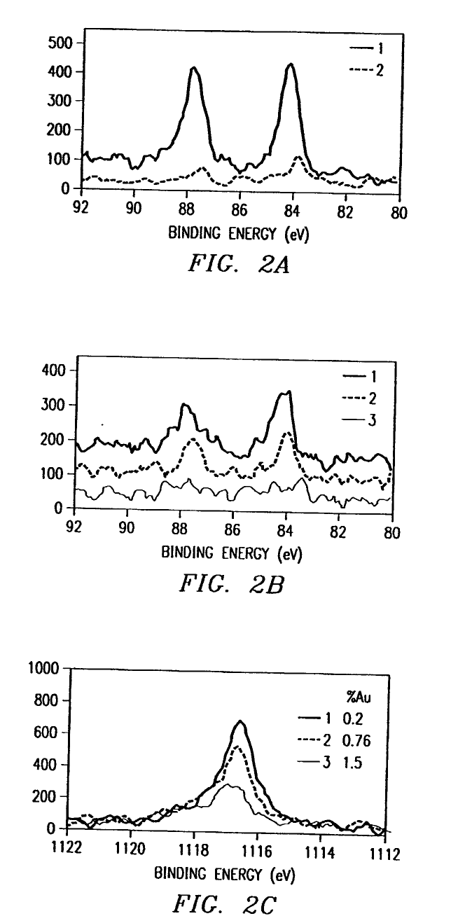

Phage, tagged with streptavidin-labeled 20-nm colloidal gold particles bound to the phage through a biotinylated antibody to the M13 coat protein, were used for quantitative assessment of specific binding. X-ray photoelectron spectroscopy (XPS) elemental composition determination was performed, monitoring the phage substrate interaction through the intensity of the gold 4f-electron signal (FIGS. 2a-c). Without the presence of the G1-3 phage, XPS confirmed that the antibody and the gold streptavidin did not bind to the GaAs(100) substrate. The gold-streptavidin binding was, therefore, specific to the peptide expressed on the phage and an indicator of the phage binding to the substrate. Using XPS it was also found that the G1-3 sequence isolated from GaAs(100) bound specifically to GaAs(100) but not to Si(100) (see FIG. 2a). In a complementary fashion the S1 clone, screened against the (100) Si surface, showed poor binding to the (100) GaAs surface.

Some GaAs sequences also bound the surface of InP (100), another zinc-blende structure. The basis of the selective binding, whether it is chemical, structural or electronic, is still under investigation. In addition, the presence of native oxide on the substrate surface may alter the selectivity of peptide binding.

The preferential binding of the G1-3 clone to GaAs(100), over the (111)A (gallium terminated) or (111)B (arsenic terminated) face of GaAs was demonstrated (FIG. 2b, c). The G1-3 clone surface concentration was greater on the (100) surface, which was used for its selection, than on the gallium-rich (111)A or arsenic-rich (111)B surfaces. These different surfaces are known to exhibit different chemical reactivities, and it is not surprising that there is selectivity demonstrated in the phage binding to the various crystal faces. Although the bulk termination of both 111 surfaces give the same geometric structure, the differences between having Ga or As atoms outermost in the surface bilayer become more apparent when comparing surface reconstructions. The composition of the oxides of the various GaAs surfaces is also expected to be different, and this in turn may affect the nature of the peptide binding.

The intensity of Ga 2p electrons against the binding energy from substrates that were exposed to the G1-3 phage clone is plotted in 2c. As expected from the results in FIG. 2b, the Ga 2p intensities observed on the GaAs (100), (111)A and (111)B surfaces are inversely proportional to the gold concentrations. The decrease in Ga 2p intensity on surfaces with higher gold-streptavidin concentrations was due to the increase in surface coverage by the phage. XPS is a surface technique with a sampling depth of approximately 30 angstroms; therefore, as the thickness of the organic layer increases, the signal from the inorganic substrate decreases. This observation was used to confirm that the intensity of gold-streptavidin was indeed due to the presence of phage containing a crystal specific bonding sequence on the surface of GaAs. Binding studies were performed that correlate with the XPS data, where equal numbers of specific phage clones were exposed to various semiconductor substrates with equal surface areas. Wild-type clones (no random peptide insert) did not bind to GaAs (no plaques were detected). For the G1-3 clone, the eluted phage population was 12 times greater from GaAs(100) than from the GaAs(111)A surface.

The G1-3, G12-3 and G7-4 clones bound to GaAs(100) and InP(100) were imaged using atomic force microscopy (AFM). The InP crystal has a zinc-blende structure, isostructural with GaAs, although the In-P bond has greater ionic character than the GaAs bond. The 10-nm width and 900-nm length of the observed phage in AFM matches the dimensions of the M13 phage observed by transmission electron microscopy (TEM), and the gold spheres bound to M13 antibodies were observed bound to the phage (data not shown). The InP surface has a high concentration of phage. These data suggest that there are many factors involved in substrate recognition, including atom size, charge, polarity and crystal structure.

The G1-3 clone (negatively stained) is seen bound to a GaAs crystalline wafer in the TEM image (not shown). The data confirms that binding was directed by the modified pIII protein of G1-3, not through non-specific interactions with the major coat protein. Therefore, peptides of the present invention may be used to direct specific peptide-semiconductor interactions in assembling nanostructures and heterostructures (FIG. 4).

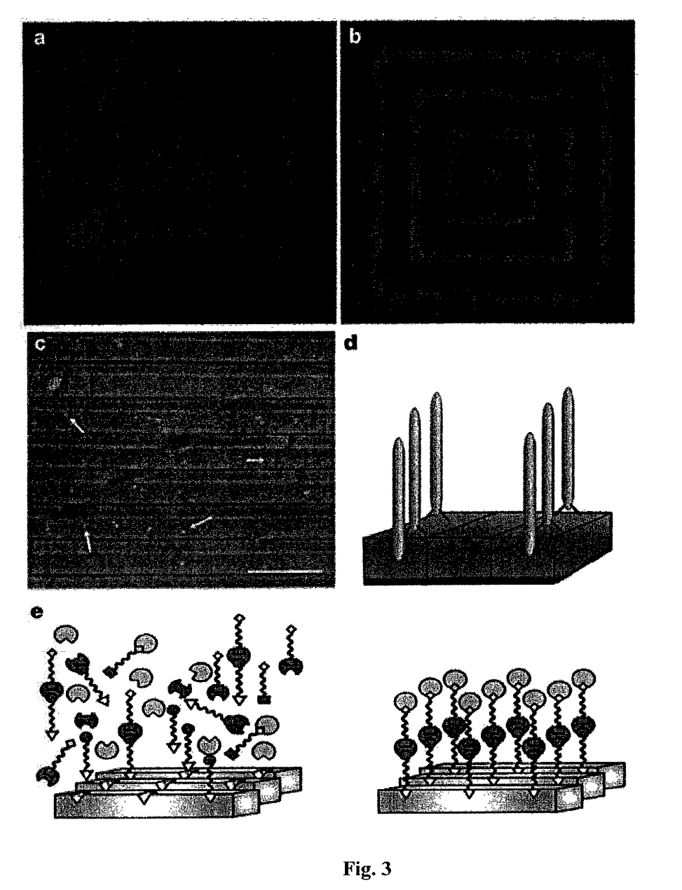

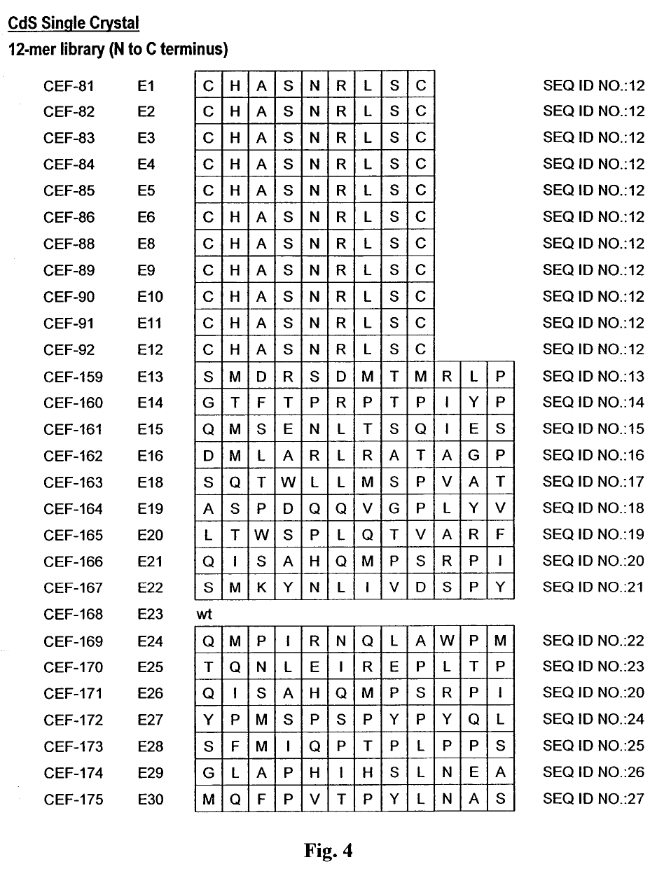

X-ray fluorescence microscopy was used to demonstrate the preferential attachment of phage to a zinc-blende surface in close proximity to a surface of differing chemical and structural composition. A nested square pattern was etched into a GaAs wafer; this pattern contained 1-μm lines of GaAs, and 4-μm SiO₂ spacings in between each line (FIGS. 3a, 3b). The G12-3 clones were interacted with the GaAs/SiO2 patterned substrate, washed to reduce non-specific binding, and tagged with an immuno-fluorescent probe, tetramethyl rhodamine (TMR). The tagged phage were found as the three red lines and the center dot, in FIG. 3b, corresponding to G12-3 binding only to GaAs. The SiO₂ regions of the pattern remain unbound by phage and are dark in color. This result was not observed on a control that was not exposed to phage, but was exposed to the primary antibody and TMR (FIG. 3a). The same result was obtained using non-phage bound G12-3 peptide.

The GaAs clone G12-3 was observed to be substrate-specific for GaAs over AlGaAs (FIG. 3c). AlAs and GaAs have essentially identical lattice constraints at room temperature, 5.66 A° and 5.65 A°, respectively, and thus ternary alloys of AlxGa1-xAs can be epitaxially grown on GaAs substrates. GaAs and AlGaAs have zinc-blende crystal structures, but the G12-3 clone exhibited selectivity in binding only to GaAs. A multilayer substrate was used, consisting of alternating layers of GaAs and of Al_{0.98}Ga_{0.02}As. The substrate material was cleaved and subsequently reacted with the G12-3 clone.

The G12-3 clones were labeled with 20-nm gold-streptavidin nanoparticles. Examination by scanning electron microscopy (SEM) shows the alternating layers of GaAs and Al_{0.98}Ga_{0.02}As within the heterostructure (FIG. 3c). X-ray elemental analysis of gallium and aluminum was used to map the gold-streptavidin particles exclusively to the GaAs layers of the heterostructure, demonstrating the high degree of binding specificity for chemical composition. In FIG. 3d, a model is depicted for the discrimination of phage for semiconductor heterostructures, as seen in the fluorescence and SEM images (FIGS. 3a-c).

The present invention demonstrates the powerful use of phage-display libraries to identify, develop and amplify binding between organic peptide sequences and inorganic semiconductor substrates. This peptide recognition and specificity of inorganic crystals has been demonstrated above with GaAs, InP and Si, and has been extended to other substrates, including GaN, ZnS, CdS, Fe₃O₄, Fe₂O₃, CdSe, ZnSe and CaCO₃ using peptide libraries by the present inventors. Bivalent synthetic peptides with two-component recognition (FIG. 4) are currently being designed; such peptides have the potential to direct nanoparticles to specific locations on a semiconductor structure. These organic and inorganic pairs and potentially multivalent templates should provide powerful building blocks for the fabrication of a new generation of complex, sophisticated electronic structures.

### EXAMPLE I

### Peptide Creation, Isolation, Selection and Characterization

Peptide selection. The phage display or peptide library was contacted with various materials such as a semiconductor crystal in Tris-buffered saline (TBS) containing 0.1% TWEEN-20, to reduce phage-phage interactions on the surface. After rocking for 1 h at room temperature, the surfaces were washed with 10 exposures to Tris-buffered saline, pH 7.5, and increasing TWEEN-20 concentrations from 0.1% to 0.5% (v/v) as selection rounds progressed. The phage were eluted from the surface by the addition of glycine-HCl (pH 2.2) for 10 minutes to disrupt binding. The eluted phage solution was then transferred to a fresh tube and then neutralized with Tris-HCl (pH 9.1). The eluted phage were titred and binding efficiency was compared.

The phage eluted after third-round substrate exposure were mixed with their Escherichia coli ER2537 or ER2738 host and plated on LB XGal/IPTG plates. Since the library phage were derived from the vector M13mp19, which carries the laczα gene, phage plaques were blue in color when plated on media containing Xgal (5-bromo-4-chloro-3-indoyl-β-D-galactoside) and IPTG (isopropyl-β-D-thiogalactoside). Blue/white screening was used to select phage plaques with the random peptide insert. Plaques were picked and DNA sequenced from these plates.

Substrate preparation. Substrate orientations were confirmed by X-ray diffraction, and native oxides were removed by appropriate chemical specific etching. The following etches were tested on GaAs and InP surfaces: NH₄OH:H₂O 1:10, HCl:H₂O 1:10, H₃PO₄:H₂O₂:H₂O 3:1:50 at 1 minute and 10 minute etch times. The best element ratio and least oxide formation (using XPS) for GaAs and InP etched surfaces was achieved using HCl:H₂O for 1 minute followed by a deionized water rinse for 1 minute. However, since an ammonium hydroxide etch was used for GaAs in the initial screening of the library, this etch was used for all other GaAs substrate examples. Si(100) wafers were etched in a solution of HF:H₂O 1:40 for one minute, followed by a deionized water rinse. All surfaces were taken directly from the rinse solution and immediately introduced to the phage library. Surfaces of control substrates, not exposed to phage, were characterized and mapped for effectiveness of the etching process and morphology of surfaces by AFM and XPS.

Multilayer substrates of GaAs and of Al_{0.98}Ga_{0.02} As were grown by molecular beam epitaxy onto (100) GaAs. The epitaxially grown layers were Si-doped (n-type) at a level of 5×10¹⁷ cm⁻³.

Antibody and Gold Labeling. For the XPS, SEM and AFM examples, substrates were exposed to phage for 1 h in Tris-buffered saline then introduced to an anti-fd bacteriophage-biotin conjugate, an antibody to the pIII protein of fd phage, (1:500 in phosphate buffer, Sigma) for 30 minute and then rinsed in phosphate buffer. A streptavidin/20-nm colloidal gold label (1:200 in phosphate buffered saline (PBS), Sigma) was attached to the biotin-conjugated phage through a biotin-streptavidin interaction; the surfaces were exposed to the label for 30 minutes and then rinsed several times with PBS.

X-ray Photoelectron Spectroscopy (XPS). The following controls were prepared for the XPS examples to ensure that the gold signal seen in XPS was from gold bound to the phage and not non-specific antibody interaction with the GaAs surface. The prepared (100) GaAs surface was exposed to (1) antibody and the streptavidin-gold label, but without phage, (2) G1-3 phage and streptavidin-gold label, but without the antibody, and (3) streptavidin-gold label, without either G1-3 phage or antibody.

The XPS instrument used was a Physical Electronics Phi ESCA 5700 with an aluminum anode producing monochromatic 1,487-eV X-rays. All samples were introduced to the chamber immediately after gold-tagging the phage (as described above) to limit oxidation of the GaAs surfaces, and then pumped overnight at high vacuum to reduce sample outgassing in the XPS chamber.

Atomic Force Microscopy (AFM). The AFM used was a Digital Instruments Bioscope mounted on a Zeiss Axiovert 100s-2tv, operating in tip scanning mode with a G scanner. The images were taken in air using tapping mode. The AFM probes were etched silicon with 125-mm cantilevers and spring constants of 20±100 Nm −1 driven near their resonant frequency of 200±400 kHz. Scan rates were of the order of 1±5 mms −1. Images were leveled using a first-order plane to remove sample tilt.

Transmission Electron Microscopy (TEM). TEM images were taken using a Philips EM208 at 60 kV. The G1-3 phage (diluted 1:100 in TBS) were incubated with GaAs pieces (500 mm) for 30 minute, centrifuged to separate particles from unbound phage, rinsed with TBS, and resuspended in TBS. Samples were stained with 2% uranyl acetate.

Scanning Electron Microscopy (SEM). The G12-3 phage (diluted 1:100 in TBS) were incubated with a freshly cleaved hetero-structure surface for 30 minute and rinsed with TBS. The G12-3 phage were tagged with 20-nm colloidal gold. SEM and elemental mapping images were collected using the Norian detection system mounted on a Hitachi 4700 field emission scanning electron microscope at 5 kV.

### EXAMPLE II

### Selection of Particle and Orientation Specific Peptides

It has been found that semiconductor nanocrystals exhibit size and shape-dependent optical and electrical properties may result in their potential applications in a variety of devices such as light emitting diode (LED), single electron transistor, photovoltaics, optical and magnetic memory, diagnostic markers and sensors. Control of particle size shape and phase is also critical in protective coatings, and pigments (car paints, house paints). To exploit these optical and electrical properties, it is necessary to synthesize crystallized semiconductor nanocrystals with, among other things, tailored size and shape.

The present invention includes compositions and methods for the selection and use of peptides that can: (1) recognize and bind technologically important materials with face specificity; (2) nucleate size constrained crystalline semiconductor materials; (3) control the crystallographic phase of nucleated nanoparticles; and (4) control the aspect ratio of the nanocrystals and, e.g, their optical properties.

Examples of materials used in this example were the Group II-VI semiconductors, which include materials such as: zinc sulfide, cadmium sulfide, cadmium selenium and zinc selenium. Size and crystal control could also be used with cobalt, manganese, iron oxides, iron sulfide, and lead sulfide as well as other optical and magnetic materials. Using the present invention, the skilled artisan can create inorganic-biologic material building blocks that serve as the basis for a radically new method of fabrication of complex electronic devices, optoelectronic device such as light emitting displays, optical detectors and lasers, fast interconnects, wavelength-selective switches, nanometer-scale computer components, mammalian implants and environmental and in situ diagnostics.

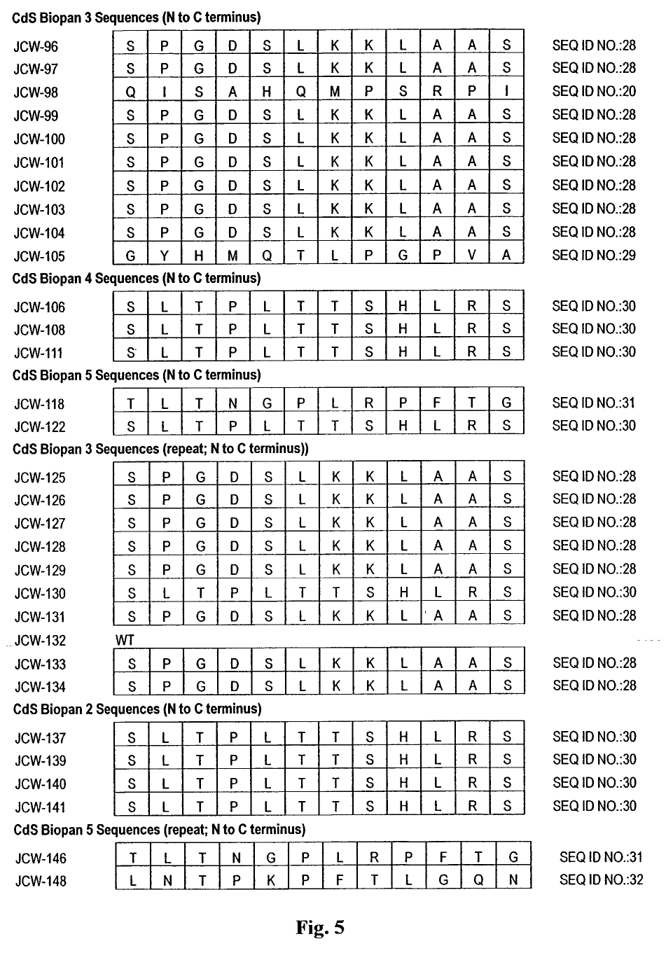

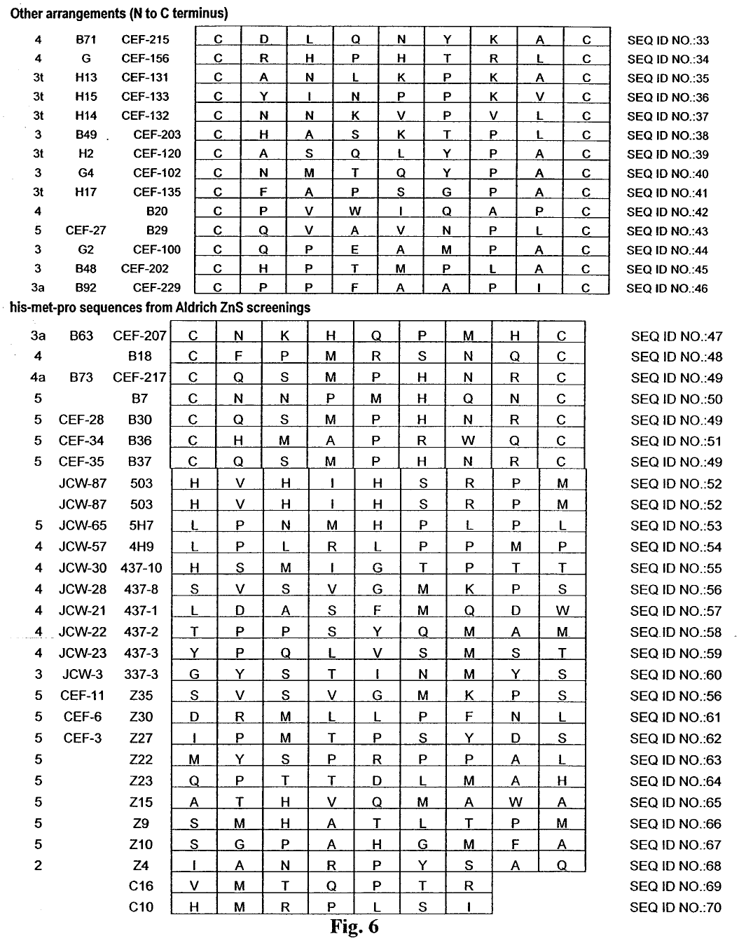

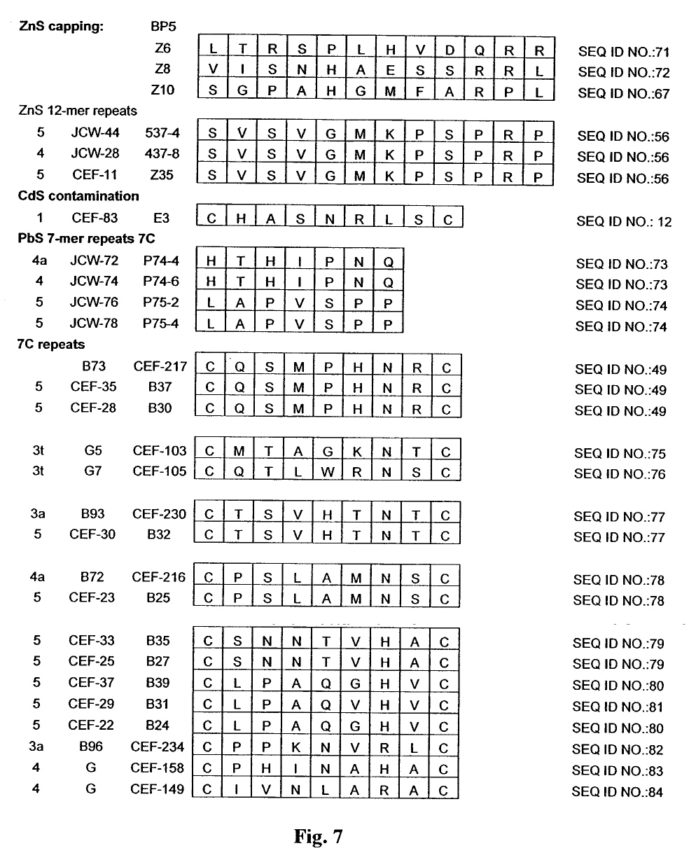

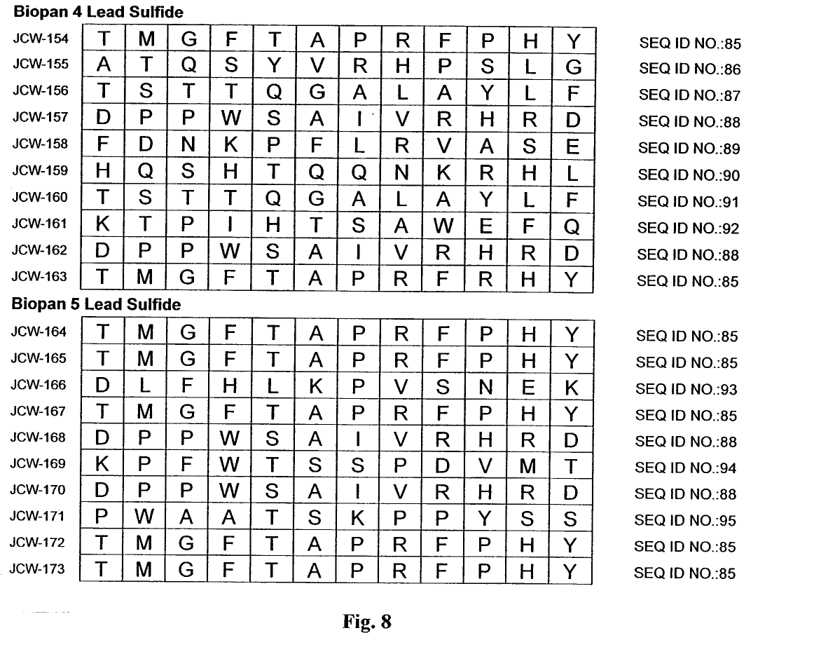

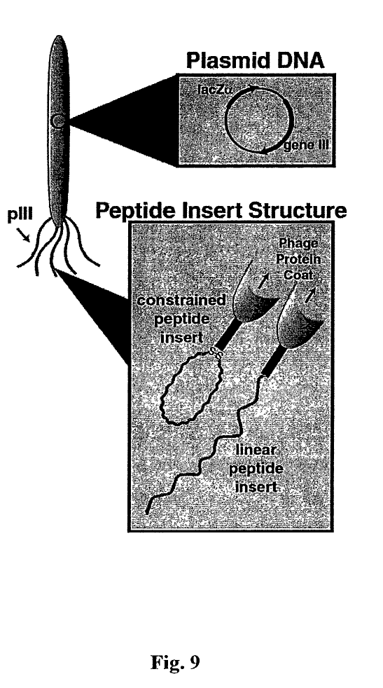

FIGS. 4-8 depict the expression of peptides using, e.g., a phage display library to express the peptides that will bind to the semiconductor material. Those of skill in the art of molecular biology will recognize that other expression systems may be used to “display” short or even long peptide sequences in a stable manner on the surface of a protein. Phage display may be used herein as an example. The phage-display library is a combinatorial library of random peptides containing between 7 and 12 amino acids. The peptides may be fused to, or form a chimera with, e.g., the pIII coat protein of M13 coliphage. The phage provided different peptides that were reacted with crystalline semiconductor structures. M13 pIII coat protein is useful because five copies of the pIII coat protein are located on one end of the phage particle, accounting for 10-16 nm of the particle. The phage-display approach provided a physical linkage between the peptide substrate interaction and the DNA that encodes that interaction. The semiconductor materials tested included ZnS, CdS, CdSe, and ZnSe.

To obtain peptides with specific binding properties, protein sequences that successfully bound to the specific crystal were eluted from the surface, amplified by, e.g., a million-fold, and reacted against the substrate under more stringent conditions. This procedure was repeated five times to select the phage in the library with the most specific binding. After, e.g., the third, fourth and fifth rounds of phage selection, crystal-specific phage were isolated and the DNA sequenced to decipher the peptide motif responsible for surface binding.

In one example of the present invention, two different peptides were found to nucleate two different phases of quantum dots. A linear 12-mer peptide, Z8, has been found that grows 3-4 nm particles of the cubic phase of zinc sulfide. A 7-mer disulfide constrained peptide, A7, has been isolated that grows nanoparticles of the hexagonal phase of ZnS. In addition, these peptides affect the aspect ratio (shape) of the nanoparticles grown. The A7 peptide has this “activity” while is it still attached to p3 of the phage or attached as a monolayer on gold. In addition phage/semiconductor nanoparticle nanowires wires were grown using an A7 fusion to the p8 protein on the virus coat. The nanoparticles grown on the phage coat show perfect crystallographic alignment of ZnS particles.

Peptides controlling nanoparticle size, morphology and aspect ratio. Phage that display a shape-controlling amino acid sequence were isolated, characterized and selected that specifically bind to ZnS, CdS, ZnSe and CdSe crystals. The binding affinity and discrimination of these peptides was tested and based on the results, peptides will be engineered for higher affinity binding. To conduct the tests, the phage library was screened against mm-size polycrystalline ZnS pieces. Binding clones were sequenced and amplified after third, fourth and fifth round selections. Sequences were analyzed and clones were tested for the ability of peptides that bind ZnS to nucleate nanoparticles of ZnS.

The clones designated Z8, A7 and Z10 clone were added to ZnS synthesis experiments to attempt to control ZnS particle size and monodispersity at room temperature in aqueous conditions. The ZnS-specific clones were interacted with Zn⁺² ions in millimolar concentrations of ZnCl₂ solution. The ZnS-specific peptide bound to the phage acts as a capping ligand, controlling crystalline particle size as ZnS is formed upon addition of Na₂S to the phage-ZnCl₂ solution.

Upon introduction of millimolar concentrations of Na₂S, crystalline material was observed to be in suspension. The suspensions were analyzed for particle size and crystal structures using transmission electron microscopy (TEM) and electron diffraction (ED). The TEM and ED data revealed that the addition of the ZnS-specific peptide bound to the phage clone affected the particle size of the forming ZnS crystals.

Crystals grown in the presence of the ZnS were observed to be approximately 5 nm in size and discrete particles. Crystals grown without the ZnS phage clones were much larger (>100 nm) and exhibited a range of sizes.

TABLE 1. Binding domains of ZnS specific clones (written amino to carboxy terminus).

A7 Asn Asn Pro Met His Gln Asn Cys (SEQ ID NO.:232)

Z8 Val Ile Ser Asn His Ala Glu Ser Ser Arg Arg Leu (SEQ ID NO.:72)

Z10 Ser Gly Pro Ala His Gly Met Phe Ala Arg Pro Leu (SEQ ID No.:233)

TABLE 2. Binding domains of CdS specific clones (written amino to carboxy terminus).

E1: Cys His Ala Ser Asn Arg Leu Ser Cys (SEQ ID NO.:12)

E14: Gly Thr Phe Thr Pro Arg Pro Thr Pro Ile Tyr Pro (SEQ ID NO.:14)

E15: Gln Met Ser Glu Asn Leu Thr Ser Gln Ile Glu Ser (SEQ ID NO.:15)

JCW-96: Ser Pro Gly Asp Ser Leu Lys Lys Leu Ala Ala Ser (SEQ ID NO.:28)

JCW-106: Ser Leu Thr Pro Leu Thr Thr Ser His Leu Arg Ser (SEQ ID NO.:30)

JCW-137: Ser Leu Thr Pro Leu Thr Thr Ser His Leu Arg Ser (SEQ ID NO.:30)

JCW-182: Cys Thr Tyr Ser Arg Leu His Leu Cys (SEQ ID NO.:234)

JCW-201: Cys Arg Pro Tyr Asn Ile His Gln Cys (SEQ ID NO.:235)

JCW-205: Cys Pro Phe Lys Thr Ala Phe Pro Cys (SEQ ID NO.:236)

The peptide insert structure expressed during phage generation, e.g., a 12-mer linear and 7-mer constrained libraries with a disulfide bond have been used, with similar results.

Peptides selected for ZnS using a 12 amino acid linear library verses a 7 amino acid constrained loop library had a significant effect on both the crystal structure of ZnS and the aspect ratio of the ZnS nanocrystals.

High resolution lattice images of nanoparticles grown in the presence of phage displaying 12 mer linear peptides that had been selected for ZnS revealed the crystals grew 3-4 nm spheres (1:1 aspect ratio) of the cubic (zinc-blende) form of ZnS. In contrast, the 7 mer constrained peptides selected to bind ZnS grew elliptical particles and wires (2:1 aspect ratio and 8:1 aspect ratio) of the hexagonal (wurzite) form of ZnS. Thus, the nanocrystal properties could be engineered by adjusting the length and sequence of the peptide. Further, electron diffraction patterns of the crystals revealed that peptides from different clones can stabilize the two different crystal structures of ZnS. The Z8 12 mer peptide stabilized the zinc-blende structure and the A7 7 mer constrained peptide stabilized the wurzite structure.

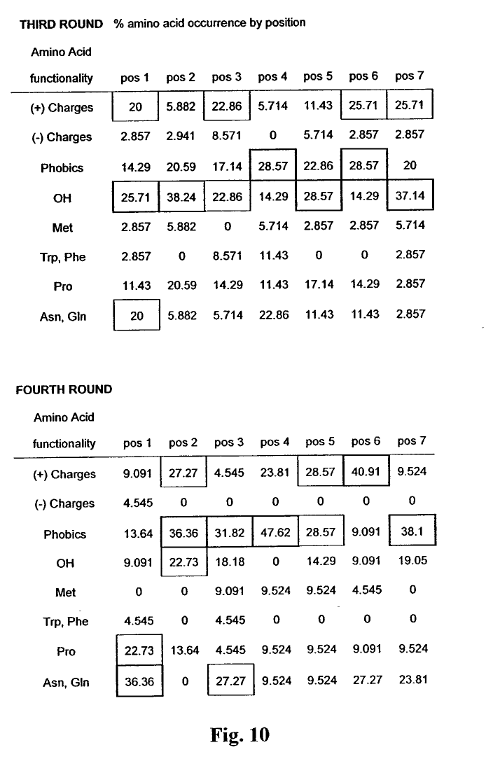

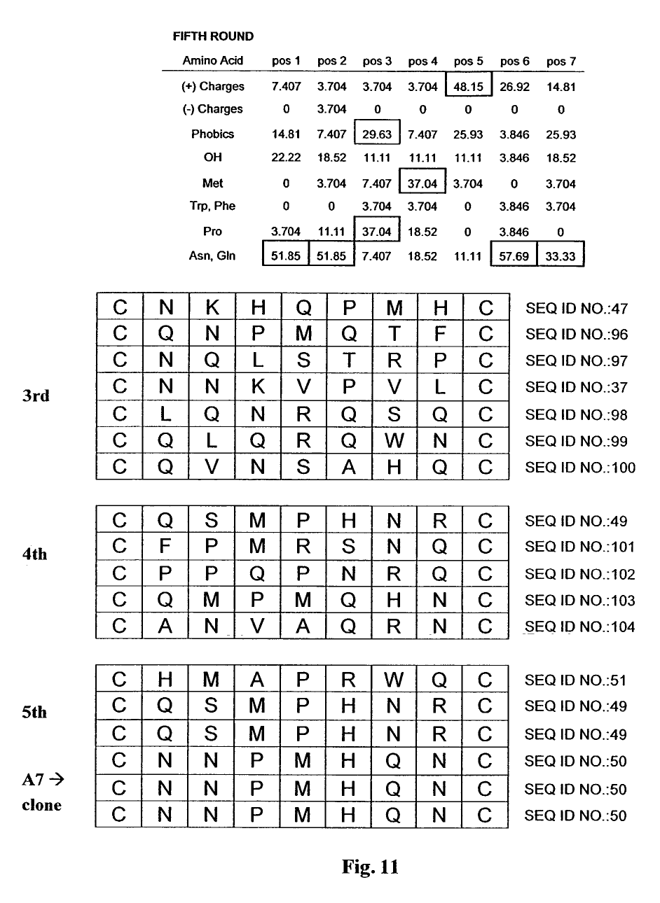

FIG. 10 shows the sequence evolution for ZnS peptides after the third, fourth and fifth rounds of selection. For peptide selection with the 7 mer constrained library, the best binding peptide sequence was obtained by the fifth round of selection. This sequence was named A7. Approximately thirty percent of the clones isolated after the fifth round of selection had the A7 sequence. The ASN/GLN at position number 7 was found to be significant starting from the third round of selection. In the fourth round of selection, the ASN/GLN also became important in position numbers 1 and 2. This importance increased in round 5. Throughout rounds 3, 4, and 5, a positive charge became prominent at position 2. FIG. 11 depicts the amino acid substitutions after the fifth round of selection in accordance with the present invention.

Site-directed mutagenesis is being conducted in the A7 sequence to test for a change in binding affinity. Mutations being tested include: position 3: his ala; position 4: met ala; position 2: gln ala; and position 6: asn ala. These changes may be made to the peptide concurrently, individually or in combinations.

The amino acid sequence motif defined for ZnS binding is, therefore (written amino to carboxy terminus): amide-amide-Xaa-Xaa-positive-amide-amide or ASN/GLN-ASN/GLN-PRO-MET-HIS-ASN/GLN-ASN/GLN (SEQ ID NO.:237).

The clones isolated for ZnS through binding studies showed preferential interaction to ZnS, the substrate against which they had been raised, versus foreign clones and foreign substrates.

Interactions of different clones with different substrates such as FeS, Si, CdS and ZnS showed that the clones isolated through binding studies for ZnS showed preferential interaction to the ZnS against which they had been raised. Briefly, after washings and infection, phage titers were counted and compared. For Z8 and Z10, no titer count was evident on any substrate except ZnS. Wild-type clones with no peptide insert were used as a control to verify that the engineered insert had indeed mediated the interaction of interest. Without the peptide, no specific binding occurred, as was evidenced by a titer count of zero.

Using the same binding method that was used for, several different ZnS clones were compared to each other. Clones having different peptide inserts at the same concentration were interacted with a similar sized piece of ZnS for one hour. The substrate-phage complex was washed repeatedly, and the bound phage was eluted by changing the pH. The eluate was infected into bacteria and the plaques were counted after an overnight incubation. Z8 showed the greatest affinity for the ZnS of the 12 mer linear peptides selected. The wild-type did not show binding to the ZnS crystal. The Z8, Z10 and the wild-type peptides did not bind to the Si, FeS or CdS crystals.

The synthesis and assembly of nanocrystals on peptide functionalized surfaces was determined. The A7 peptide was tested alone for the ability to control the structure of ZnS. The A7 peptide, which specifically selected and grew ZnS crystals when attached to the phage, was applied in the form of a functionalized surface on a gold substrate that could direct the formation of ZnS nanocrystals from solution. A process that is used to prepare self-assembled monolayer was employed to prepare a functionalized surface.

To determine the ability and selectivity of A7 in the formation of ZnS nanocrystals, different kinds of surfaces with different surface chemistry on the gold substrate were interfaced with ZnS precursor solution. ZnCl₂ and Na₂S were used as the ZnS precursor solutions. CdS precursor solution of CdCl₂ and Na₂S was used as the CdS source. The crystals that formed on the four surfaces were characterized by SEM/EDS and TEM observation.

Control surface 1 consisted of a blank gold substrate. After being aged for 70 h in either ZnS solution or CdS solution, crystals formation was not observed. Control surface 2 consisted of a 2-mercaptoethyamine self-assembled monolayer on a gold substrate. This surface could not induce the formation of ZnS and CdS nanocrystals. In a few places, ZnS precipitates were observed. For the CdS system, sparsely distributed 2 micron CdS crystals were observed. Precipitation of these crystals occurred when the concentrations of both Cd⁺² and S⁻² were at 1×10⁻³ M.

The third surface tested was an A7-only functionalized gold surface. This surface was able to direct the formation of 5 nm ZnS nanocrystals, but could not direct the formation of CdS nanocrystals.

The fourth surface tested was an A7-amine functionalized gold surface that was prepared by aging control surface 2 in A7 peptide solution. The ZnS crystals formed on this surface were 5 nm and the CdS crystals were 1-3 μm. The CdS crystals could also be formed on the amine-only surface.

From the results of the four surfaces, the A7 peptide could direct the formation of ZnS nanocrystals for which it was selected, but could not direct the formation of CdS nanocrystals. Further, peptides selected against CdS could nucleate nanoparticles of CdS.

The peptides that could specifically nucleate semiconductor materials were expressed on the p8 major coat protein of M13. The p8 proteins are known to self-assemble into a highly oriented, crystalline protein coat. The hypothesis was that if the peptide insert could be expressed in high copy number, the crystalline structure of the p8 protein would be transferred to the peptide insert. It was also predicted that if the desired peptide insert maintained a crystal orientation relative to the p8 coat, then the crystals that nucleated from this peptide insert should grow nanocrystals that are crystallographically related. This prediction was tested and confirmed using high resolution TEM.

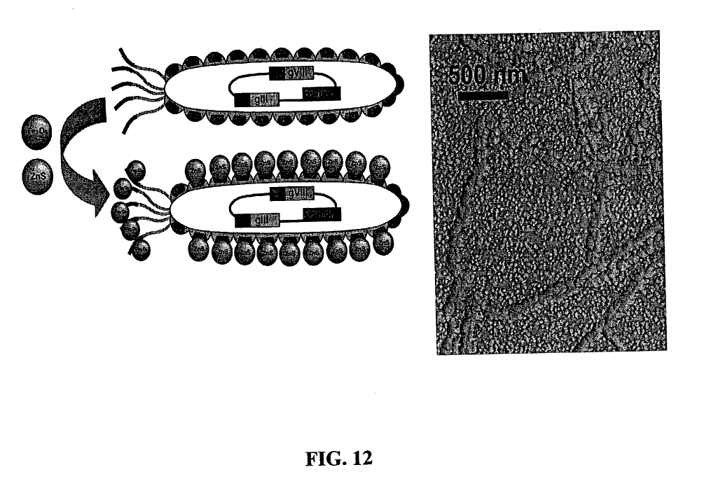

FIG. 12 shows a schematic diagram of the p8 and p3 inserts used to form nanowires. ZnS nanowires were made by nucleating ZnS nanoparticles off of the A7 peptide fusion along the p8 protein coat of M13 phage. The ZnS nanoparticles coated the surface of the phage. The HR TEM image of ZnS nucleated on the coats of M13 phage that have the A7 peptide insert within the p8 protein showed that the nanocrystals nucleated on the coat of the phage were perfectly oriented. It is not clear whether the phage coat was a mixture of the p8-A7 fusion coat protein and the wild-type p8 protein. Similar experiments were performed with the Z8 peptide insert, and although the ZnS crystals were also nucleated along the phage, they were not orientated relative to each other.

Atomic force microscopy (AFM) was used to imagine the results, which indicated that the p8-A7 self-assembling crystals coated the surface of the phage, creating nanowires along the crest of the chimeric protein at the location of the A7 peptide sequence (data not shown). Nanowires were made by nucleating ZnS nanoparticles at the sites of the p8-A7 fusion along the coat of M13.

Nanocrystal nucleation of ZnS on the coat M13 phage that have the A7 peptide insert in the p8 protein was confirmed by high resolution TEM. Crystal nucleation was achieved despite the fact that some wild type p8 protein was found mixtured in with the p8-A7 fusion coat protein. The nanocrystals nucleated on the coat of the phage were perfectly orientated, as evidenced by lattice imaging (data not shown). The data demonstrates that peptides can be displayed in the major coat protein with perfect orientation conservation, and that these orientated peptides can nucleate orientated mondispersed ZnS semiconductor nanoparticles.

The cumulative data showed that some peptides could be displayed in the major coat protein with perfect orientation conservation and that these peptides could nucleate orientated ZnS semiconductor nanoparticles.

Peptide selection. The phage display or peptide library was contacted with the semiconductor, or other crystals, in Tris-buffered saline (TBS) containing 0.1% TWEEN-20, to reduce phage-phage interactions on the surface. After rocking for 1 hour at room temperature, the surfaces were washed with 10 exposures to Tris-buffered saline, pH 7.5, and increasing TWEEN-20 concentrations from 0.1% to 0.5% (v/v) as selection rounds progressed. The phage display was eluted from the surface by the addition of glycine-HCl (pH 2.2) for 10 minutes to disrupt binding. The eluted phage solution was then transferred to a fresh tube and then neutralized with Tris-HCl (pH 9.1). The eluted phage were titred and binding efficiency was compared.

The phage eluted after the third-round of substrate exposure were mixed with an Escherichia coli ER2537 or ER2738 host and plated on Luria-Bertani (LB) XGal/IPTG plates. Since the library phage were derived from the vector M13mp19, which carries the lacZα gene, phage plaques, or infection events, were blue in color when plated on media containing Xgal (5-bromo-4-chloro-3-indoyl-β-D-galactoside) and IPTG (isopropyl-β-D-thiogalactoside). Blue/white screening was used to select phage plaques with the random peptide insert. DNA from these plaques was isolated and sequenced.

Atomic Force Microscopy (AFM). The AFM used was a Digital Instruments Bioscope mounted on a Zeiss Axiovert 100s-2tv, operating in tapping mode. The images were taken in air using tapping mode. The AFM probes were etched silicon with 125-mm cantilevers and spring constants of 20±100 Nm⁻¹ driven near their resonant frequency of 200±400 kHz. Scan rates were of the order of 1±5 mms⁻¹. Images were leveled using a first-order plane to remove sample tilt.

Transmission Electron Microscopy (TEM). TEM images were taken on JEOL 2010 and JEOL200CX transmission electron microscopes. The TEM grids used were carbon on gold. No stain was used. After the samples were grown, the reaction mixture was concentrated on molecular weight cut-off filters and washed four times with sterile water to wash away any excess ions or non-phage bond particles. After concentrating to 20-50 μl, the sample was then dried down on TEM or AFM specimen grids.

### EXAMPLE III

### Biologic Materials with Affinities for Elemental Carbon-Containing Molecules

In this example, seven- and twelve-mer peptide sequences with affinities to carbon planchets, highly ordered pyrolytic graphite (HOPG), and single-walled nanotube (SWNT) paste were determined using phage display. Among the phage clones selected from biopanning, clones Graph5-01 (N′-WWSWHPW-C′) (SEQ ID NO:238) and Graph53-01 (N′-HWSWWHP-C′) (SEQ ID NO:239) bound with greatest efficiencies to carbon planchets in phage binding studies. Clone Hipco12R44-01 (N′-DMPRTTMSPPPR-C′) (SEQ ID NO:196) bound best to SWNT paste.

The relative abilities of these phage to bind to their corresponding substrates was verified by labeling the phage with fluorescein-labeled anti-M13 phage antibodies and visualizing them on their substrates using confocal microscopy. Confocal microscopy was also used to visualize the binding of the substrates to fluorescently-labeled synthetic peptides containing these substrate-specific sequences. Clone Graph5-01 displayed some crossreactivity to HOPG, as determined by AFM. Examples of additional methodology is described below.

Biopanning. Carbon planchetts (obtained from Ted Pella, Inc., with dimensions at about 12.7 mm diam×1.6 mm thick; in pieces at about 5×2×1.6 mm) and highly ordered pyrolytic graphite (HOPG) (obtained from the University of Texas at Austin) were used as graphite sources for biopanning. SWNT paste was molded into cigar-shaped aggregates (at least about 0.1 g wet) and dessicated for at least about one night before use in biopanning (final dried mass was at about 0.05 g). PhD-C7C and PhD-12mer libraries were obtained from New England Biolabs, Inc. (Beverly, Mass.), and biopanning was performed according to manufacturer instructions. Biopanning for each substrate was repeated at least once.

Phage Clone Nomenclature. The names of phage clones selected against carbon planchets were prefaced by “Graph.” Phage clones selected against SWNT paste were prefaced by “Hipco.” Phage clones selected against HOPG were prefaced by “HOPG.” Selected clones with 12-mer inserts were named, (Substrate)12R(round#)(round repeat#)-(SEQ ID NO:); whereas clones with constrained 7-mer inserts were named, (Substrate)(round#)(round repeat#)-(SEQ ID NO:).

Peptides. The biotinylated peptide Hipco2B (N′-DMPRTTMSPPPRGGGK-C′-biotin) (SEQ ID NO.:244) was synthesized by Genemed Synthesis, Inc. (San Francisco, Calif.). Biotinylated peptides Graphite1B (N′-ACWWSWHPWCGGGK-C′-biotin) (SEQ ID NO:240), JH127B (N′-ACDSPHRHSCGGGK-C′-biotin)(SEQ ID NO:241), and JH127MixB (N′-ACPRSSHDHCGGGK-C′-biotin) (SEQ ID NO:242) were synthesized by the ICMB Protein Microanalysis Facility (University of Texas at Austin) and purified by reversed phase HPLC (HiPore RP318 250×10 mm column, BioRad, Hercules, Calif., acetonitrile gradient). Disulfide bond formation between the cysteines of the Graphite1B peptide was performed by iodine oxidation according to methods known in the art of chemistry, resulting in the cyclized Graphite1B peptide. The purity and molecular masses of the peptides were verified using electrospray ionization mass spectrometry (Esquire-LC00113, Bruker Daltonics, Inc., Billerica, Mass.).

Phage Binding Studies. Dessicated, flat, square-shaped aggregates of SWNT paste (at least about 0.05 g wet and 0.0025 g dried) and at least about 0.04 g carbon planchet pieces were used for binding studies. Phage clones were amplified and titered (according to phage library manufacturer instructions) at least twice before use. Equal amounts (at least about 5×10¹⁰ pfu) of each phage clone were separately incubated with the SWNT/carbon planchet (e.g., as aggregates) in 1 ml TBS-T [50 mM Tris, 150 mM NaCl, pH 7.5, 0.1% Tween-20] for 1 hour at room temperature with rocking in a microcentrifuge tube. The aggregate surfaces were then washed 9-10 times with TBS-T (1 ml per wash), and phage were eluted off the surfaces by exposure to 0.5 ml 0.2 M Glycine HCl (pH 2.2) for 8 minutes. The eluted phage were immediately transferred to a fresh tube, neutralized with 0.15 ml 1 M Tris HCl (pH 9.1), and then titered in duplicate. Each binding experiment was performed twice. In one embodiment of the present invention, repeated binding studies using SWNT aggregates using the same aggregates (ones used for the original experiments) included an initial wash with 1 ml 100% ethanol for 1 hour and then twice with 1 ml water).

Confocal Microscopy. Phage clones were amplified and titered (according to phage library manufacturer instructions) at least twice before use. Equal amounts (5×10⁹ pfu) of each phage clone were separately incubated with pieces of carbon planchet or small amounts of wet SWNT paste in 0.2-0.3 ml TBS-T for 1 hour in a microcentrifuge tube with occasional shaking. The carbon planchet/SWNT aggregate(s) were then washed twice with TBS-T (1 ml per wash), incubated for 45 minutes with 0.2-0.3 ml of biotinylated mouse monoclonal anti-M13 antibody (1:100 dilution in TBS-T, Exalpha Biologicals, Inc., Boston, Mass.). The aggregates were then washed twice with TBS-T (1 ml per wash), incubated for 10 minutes with 0.2-0.3 ml streptavidin-fluorescein (1:100 dilution in TBS-T from Amersham Pharmacia Biotech, Uppsala, Sweden), and then washed twice with TBS-T (1 ml per wash). Excess fluid was then removed from the aggregates. The SWNT paste was resuspended in Gel/Mount (Biomedia Corp., Foster City, Calif.) and mounted on a glass slide with a No. 1 coverslip. The carbon planchets were mounted on a glass slide with vacuum grease, covered with Gel/Mount, and topped with a coverslip. For the SWNT paste samples, centrifugation was required for each labeling and washing step.

Peptides (at least about 1 mg/ml) were separately incubated with pieces of carbon planchet or small amounts of wet SWNT paste in 0.15 ml TBS-T for 1 hour in a microcentrifuge tube with occasional shaking. Original 10 mg/ml stocks of Hipco2B were found to be soluble in 55% acetonitrile and cyclized and noncyclized Graphite1B in 45% acetonitrile. Upon dilution in TBS-T, these peptides formed white precipitates. The substrates were then washed 2-3 times with TBS-T (1 ml per wash), incubated for 15 minutes with 0.15 ml streptavidin-fluorescein (1:100 dilution in TBS), and then washed 2-3 times with TBS (1 ml per wash). Excess fluid was removed from the substrates. The SWNT paste was resuspended in Gel/Mount and mounted on a glass slide with a coverslip. The carbon planchets were mounted on a glass slide with vacuum grease, covered with Gel/Mount, and topped with a coverslip. For the SWNT paste samples, centrifugation was required for each labeling and washing step.

Confocal images were obtained on a Leica TCS 4D Confocal Microscope (ICMB Core Facility, University of Texas at Austin). Images were presented as maximum intensity composites.

AFM. Phage clones were amplified and titered (according to phage library manufacturer instructions) at least twice before use. Equal amounts (5×10⁹ pfu) of each phage clone were separately incubated with freshly cleaved layers of HOPG in 2 ml TBS for 1 hour with rocking in 35 mm×10 mm petri dishes. The substrates were then transferred to microcentrifuge tubes, washed twice with water (1 ml per wash), and dessicated overnight. Images were taken in air using tapping mode on a Multimode Atomic Force Microscope (Digital Instruments, Santa Barbara, Calif.).

Biopanning Sequences. M13 phage libraries with 12-mer and constrained 7-mer sequences inserted into their pIII coat protein were used to select clones with specificities toward carbon planchets, HOPG, and SWNT paste.

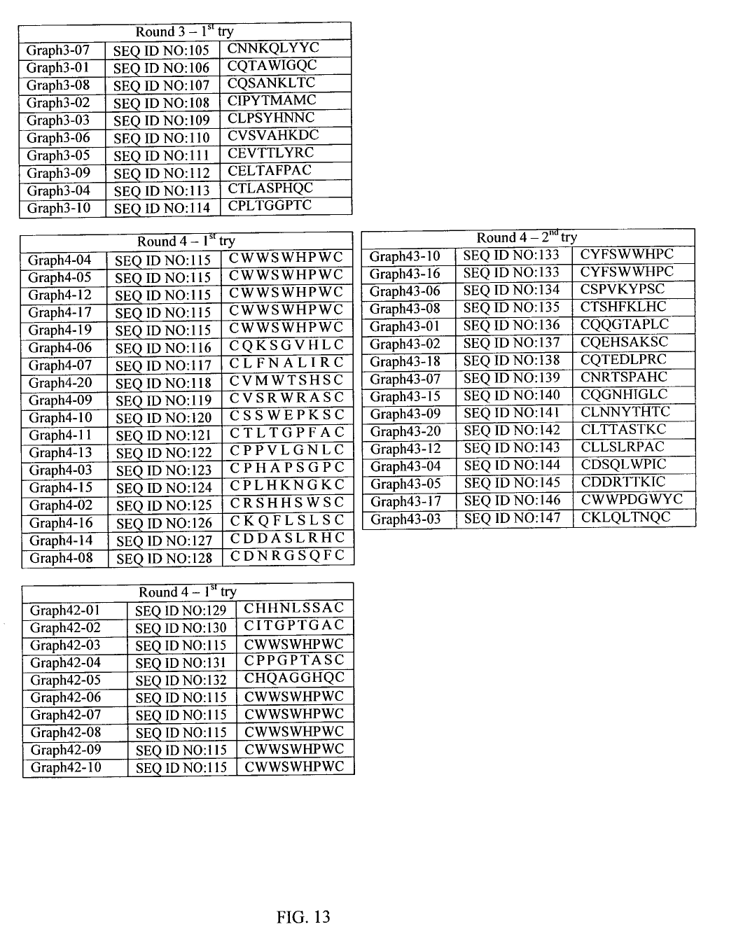

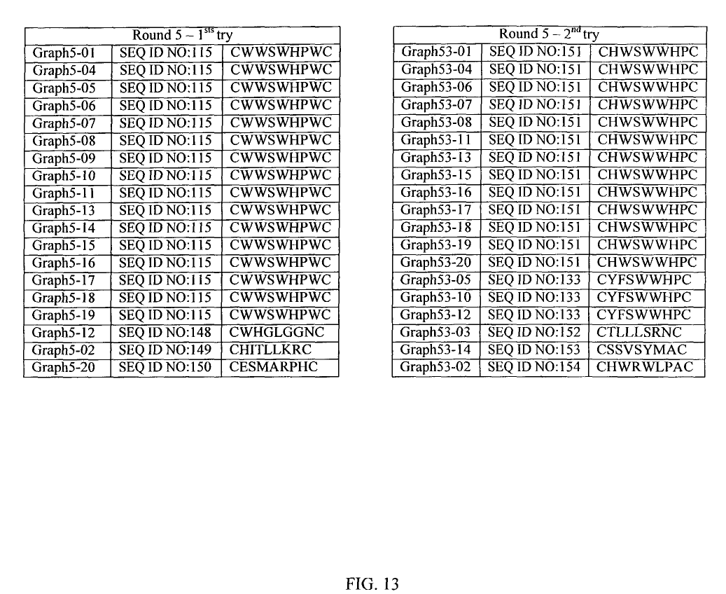

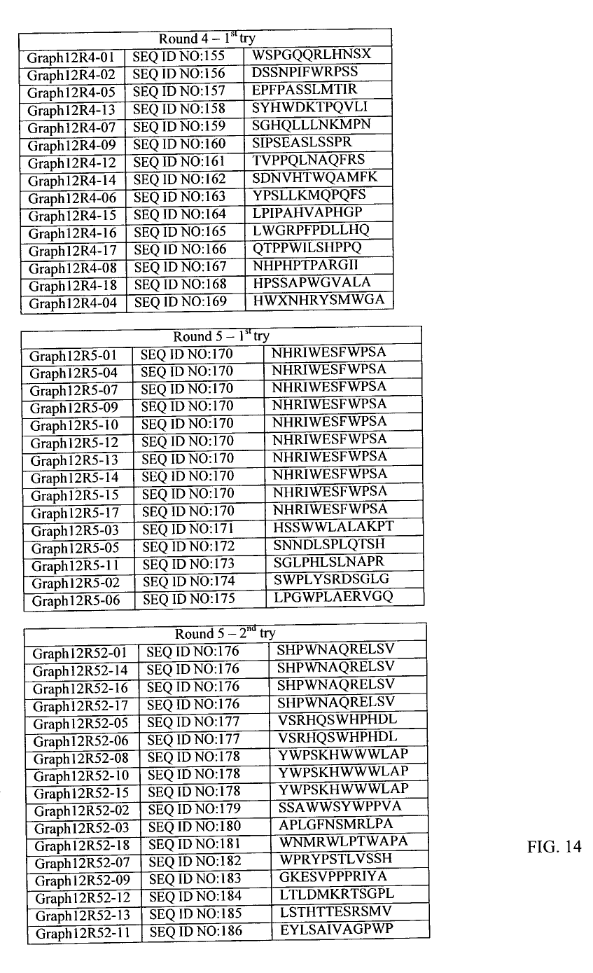

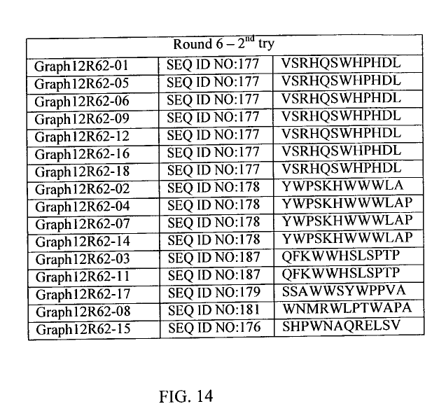

For Carbon Planchet. Selection using the PhD-C7C library against carbon planchets yielded a dominant phage clone with the peptide insert sequence N′-WWSWHPW-C′ (SEQ ID NO:238) by the 4th round as shown in FIG. 13. Upon repeating the selection, a similar dominant sequence N′-HWSWWHP-C′ (SEQ ID NO:239) and a less dominant sequence N′-YFSWWHP-C′ (SEQ ID NO:243) were obtained by the 4th round. Selection with the PhD-12 library yielded the consensus sequence N′-NHRIWESFWPSA-C′ (SEQ ID NO:172) by the 5th round, and repeating the selection yielded the sequences N′-VSRHQSWHPHDL-C′ (SEQ ID NO:179) and N′-YWPSKHWWWLAP-C′ (SEQ ID NO:180) by the 6th round, as indicated in FIG. 14. These sequences were rich in aromatic residues and commonly included the residues S, W, H, and P. One one embodiment of the present invention, N′-SHPWNAQRELSV-C′ (SEQ ID NO:178) was observed in round 5 of selection with the PhD-12 library, but was a contaminating sequence from biopanning against SWNT paste; the sequence disappeared in subsequent rounds.)

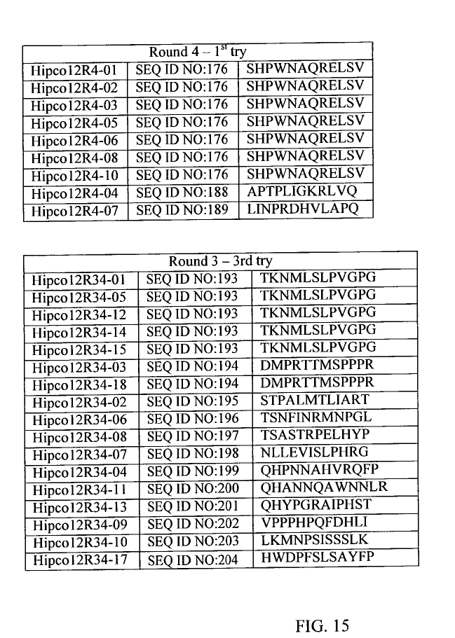

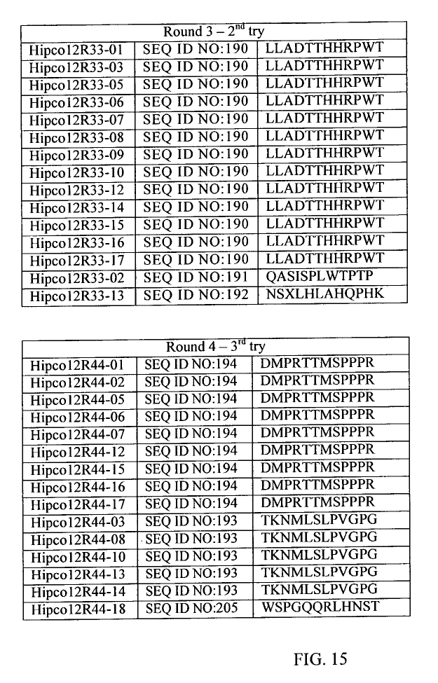

For SWNT Paste. Biopanning with the PhD-C7C library against SWNT paste was unsuccessful due to the domination of the selected phage by the “wildtype” phage clone (containing no peptide insert in pIII). As shown in FIG. 15, the consensus sequence N′-SHPWNAQRELSV-C′ (SEQ ID NO:178) was obtained by selection using the PhD-12 library by the 4th round, and second and third repeats of the selection process yielded the sequences N′-LLADTTHHRPWT-C′ (SEQ ID NO:192), N′-DMPRTTMSPPPR-C′ (SEQ ID NO:196), and N′-TKNMLSLPVGPG-C′ (SEQ ID NO:195).

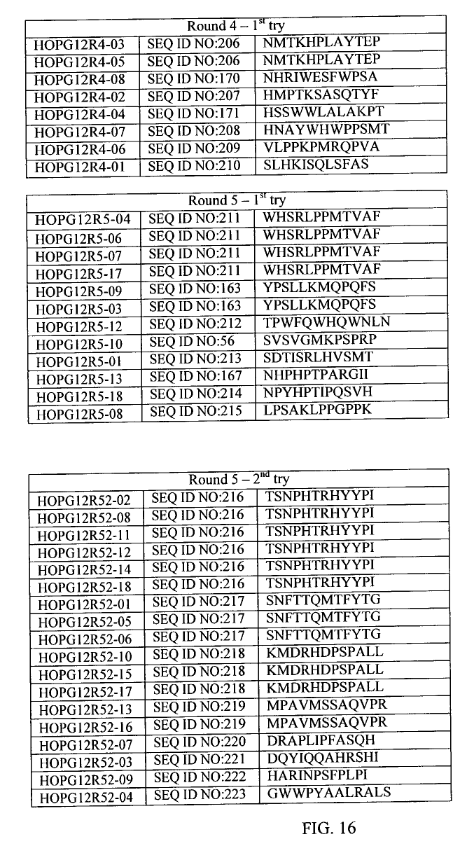

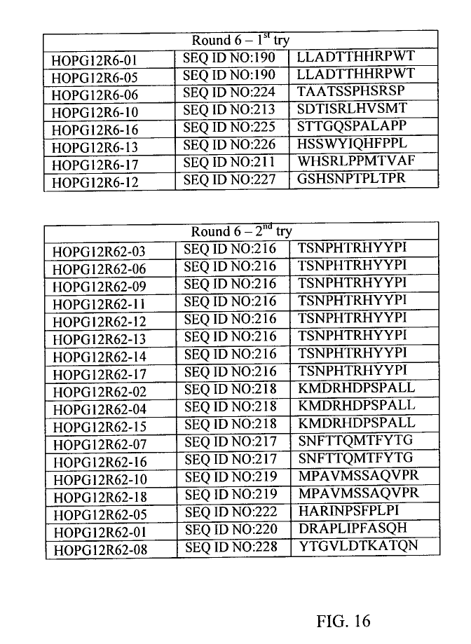

For HOPG. Selection against HOPG using the PhD-C7C library was not performed, but the PhD-12 library yielded the dominant sequence N′-TSNPHTRHYYPI-C′ (SEQ ID NO:219) and the less dominant sequences N′-KMDRHDPSPALL-C′ (SEQ ID NO:221) and N′-SNFTTQMTFYTG-C′ (SEQ ID NO:220) by the 5th round as shown in FIG. 16. (NOTE: The sequence N′-LLADTTHHRPWT-C′ (SEQ ID NO:192) was also observed in the first selection but was found to be a contaminating sequence from biopanning against SWNT paste.)

An example of many major sequences obtained from biopanning is presented in TABLE 3.

TABLE 3
Example of consensus sequences (N′-to C′-terminus)
obtained from biopanning

| Li- | | | |
|---|---|---|---|
| brary | Carbon Planchet | SWNT Paste | HOPG |
| PhD- | WWSWHPW | Unsuccessful | Not performed |
| C7C | (SEQ ID NO:238) | | |
| | HWSWWHP | | |
| | (SEQ ID NO:239) | | |
| | YFSWWHP | | |
| | (SEQ ID NO:243) | | |
| PhD- | NHRIWESFWPSA | SHPWNAQRELSV | TSNPHTRHYYPI |
| 12 | (SEQ ID NO:245) | (SEQ ID NO:178) | (SEQ ID NO:219) |
| | VSRHQSWHPHDL | LLADTTHHRPWT | KMDRHDPSPALL |
| | (SEQ ID NO:179) | (SEQ ID NO:192) | (SEQ ID NO:221) |
| | YWPSKHWWWLAP | DMPRTTMSPPPR | SNFTTQMTFYTG |
| | (SEQ ID NO:180) | (SEQ ID NO:196) | (SEQ ID NO:220) |
| | | TKNMLSLPVGPG | |
| | | (SEQ ID NO:195) | |

Phage binding studies. The relative binding efficiencies of the different phage clones determined from biopanning were tested by exposing carbon planchet pieces and SWNT paste aggregates separately to equal numbers (5×10¹⁰ pfu) of each phage clone for 1 hour and titering the amount of each clone left bound to the substrate surfaces after washing with TBS-T. Bound phage were then eluted from the substrates with 0.2 M Glycine HCl, pH 2.2 and quantified by titering. The clones used for these experiments are listed in TABLE 4. The A7 (constrained 7-mer insert) and Z8 (12-mer insert) clones and “wildtype” clone were used as negative controls.

TABLE 4
PIII inserts of phage clones used for phage binding
studies

| | Library | pIII insert (N′- to C′- |
|---|---|---|
| Phage Clone | Source | terminus) |
| Hipco12R4-01 | PhD-12 | SHPWNAQRELSV (SEQ ID NO:178) |
| Hipco12R42- | PhD-12 | LLADTTHHRPWT (SEQ ID NO:192) |
| 01 | | |
| Hipco12R44- | PhD-12 | DMPRTTMSPPPR (SEQ ID NO:196) |
| 01 | | |
| Hipco12R44- | PhD-12 | TKNMLSLPVGPG (SEQ ID NO:195) |
| 03 | | |
| Graph5-01 | PhD-C7C | WWSWHPW (SEQ ID NO:238) |
| Graph53-01 | PhD-C7C | HWSWWHP (SEQ ID NO:239) |
| Graph53-05 | PhD-C7C | YFSWWHP (SEQ ID NO:243) |
| Graph12R5-01 | PhD-12 | NHRIWESFWPSA (SEQ ID NO:245) |
| Graph12R62- | PhD-12 | VSRHQSWHPHDL (SEQ ID NO:179) |
| 01 | | |
| Graph12R62- | PhD-12 | YWPSKHWWWLAP (SEQ ID NO:180) |
| 02 | | |
| A7 | PhD-C7C | NNPHMQN (SEQ ID NO:229) |
| Z8 | PhD-12 | VISNHAESSRRL (SEQ ID NO:230) |
| Graph4-18 | PhD-12, - | no insert (“wildtype”) |
| | C7C | |

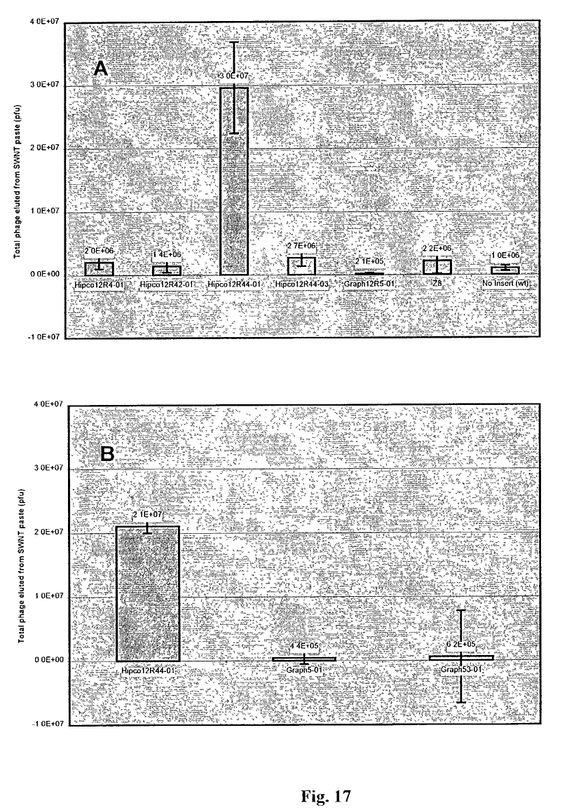

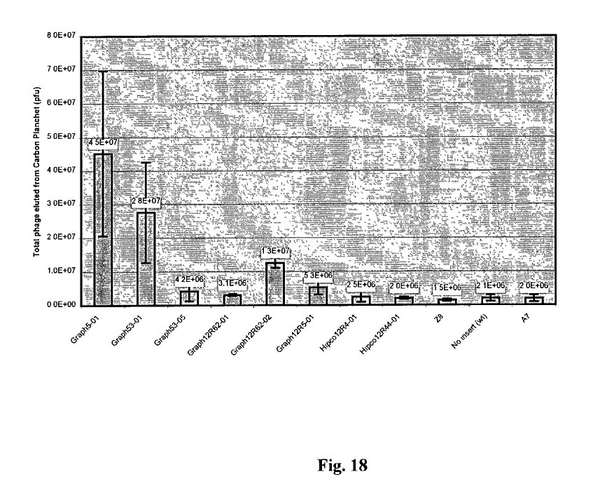

As shown in FIG. 17 (panels A and B), phage clone Hipco12R44-01 bound to SWNT paste in higher numbers than all other SWNT- or carbon planchet-specific clones, whereas clones Graph5-01 and Graph53-01, as shown in FIG. 18, bound with greatest efficiencies to carbon planchet. Little crossreactivity to SWNT paste was observed by the clones selected against carbon planchet. In addition, clones selected against SWNT paste were not crossreactive with carbon planchet.

While several consensus sequences were obtained from the biopanning process, not all of the phage clones selected by biopanning may be efficient binders (i.e., “efficient” meaning having affinities to the substrates greater than that of the wildtype clone, as determined by this type of binding or affinity study). The inability to completely remove all binding phage from the substrates using the elution buffer (0.2 M Glycine HCl, pH 2.2) in these binding studies may be a possible source of error in the interpretation of these experiments. These results may also illustrate the significance of selecting and testing several consensus sequences for each substrate (i.e., repeated biopanning may yield better sequences).

Visualization of Phage and Peptides on Substrates by Confocal Microscopy

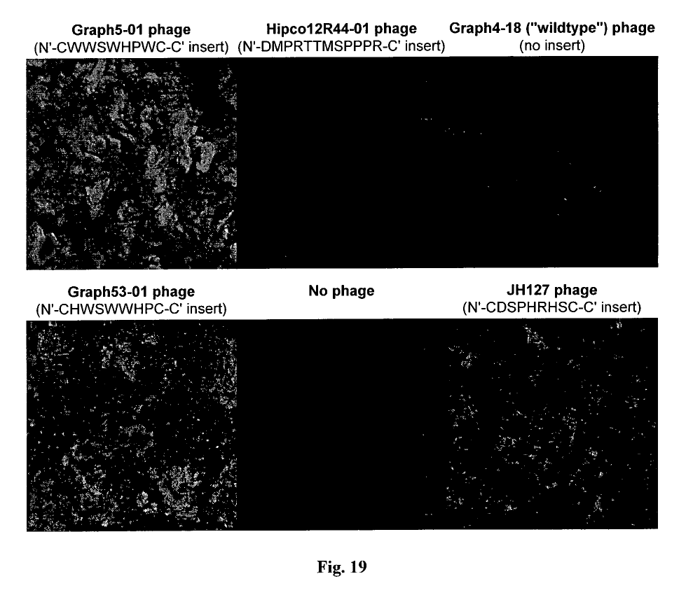

Carbon Planchet. As shown in FIG. 19, the binding of the carbon planchet-specific phage clones (Graph5-01 phage and Graph53-01 phage) to their substrates was visualized by exposing carbon planchet pieces separately to equal numbers (5×10⁹ pfu) of each clone for 1 hour, labeling the phage with a biotinylated anti-M13 antibody, labeling the antibody with streptavidin-fluorescein, and visualizing the complexes by confocal microscopy. (All images 250 μm×250 μm unless noted.) Phage clones Hipco12R44-01, JH127 (97 μm×97 μm) (from Sandra Whaley, with constrained pIII insert N′-DSPHRHS-C′) (SEQ ID NO:231), and wildtype (Graph4-18, no insert) clone were used as negative controls. Consistent with the results of the above phage binding studies, carbon planchet bound most efficiently to clone Graph5-01 and, to a lesser extent, to Graph53-01 as shown in FIG. 19. A considerable amount of crossreactivity was observed between the substrate and clone JH127, but very little binding was observed between carbon planchet and clone Hipco12R44-01 or the wildtype clone.

The binding of carbon planchet to peptides with sequences corresponding to the pIII inserts of the phage clones above was also visualized by confocal microscopy. Equal amounts (1 mg/ml) of cyclized peptide Graphite1B (corresponding to clone Graph5-01), noncyclized peptide Graphite1B, peptide Hipco2B (corresponding to clone Hipco12R44-01), peptide JH127B (corresponding to clone JH127), and peptide JH127MixB (also corresponding to clone JH127 but having a mixed amino acid sequence) were separately exposed to carbon planchet pieces for 1 hour and then labeled with streptavidin-fluorescein.

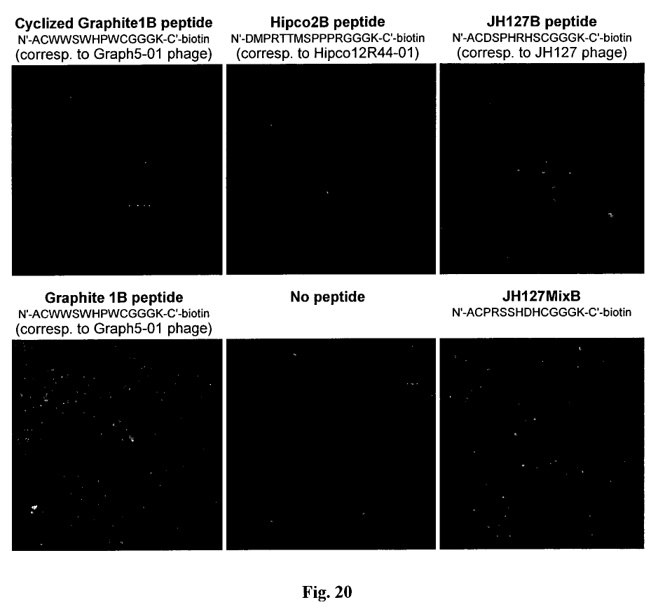

As shown in FIG. 20, a detectable amount of background fluorescence was observed in the sample incubated with no peptide, indicating that nonspecific binding occurred between the streptavidin-fluorescein and substrate. This result is most likely due to insufficient washing in this particular experiment, since a similar sample that was not exposed to phage nor peptide in the experiment depicted in FIG. 19 exhibited no background fluorescence. Despite this background fluorescence, the sample exposed to noncyclized Graphite1B exhibited a higher degree of fluorescence than the other samples. In contrast, the fluorescence displayed by the cyclized Graphite1B and Hipco2B samples was no higher than the background, indicating that the cyclization of Graphite1B interfered with substrate binding (images 250 μm×250 μm). A slightly higher degree of binding was observed between the substrate and peptides JH127B and JH127MixB. The amino acid residues common to the Graphite1B, JH127B, and JH127MixB peptides are S, P, and H. Future confocal experiments visualizing peptide binding to carbon planchet should utilize higher concentrations of peptide to enhance fluorescence and better washing procedures to decrease background.

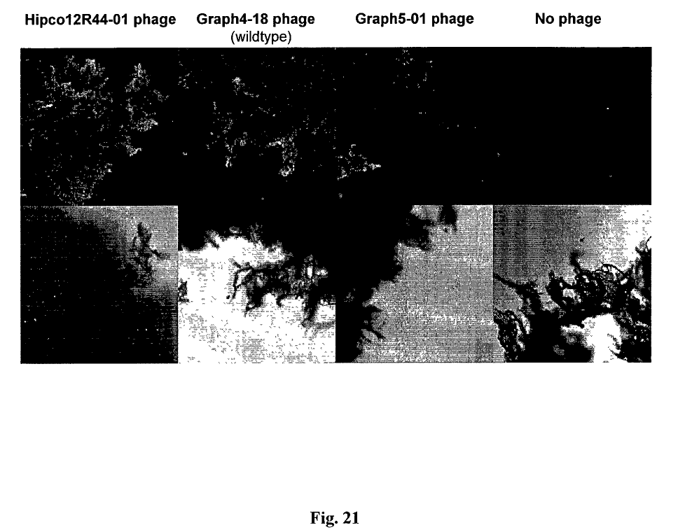

SWNT Paste. The binding of SWNT paste to the phage clone with the highest affinity to SWNT paste (Hipco12R44-01) was also visualized by confocal microscopy as shown in FIG. 21 (images 250 μm×250 μm). The Graph5-01 and wildtype (Graph4-18, no insert) clones were used as negative controls. The Hipco12R44-01 clone showed a high degree of fluorescence, but considerable fluorescence was also observed in the control samples. No background fluorescence was observed in the absence of phage, indicating that the fluorescence in the Graph5-01 and wildtype samples was not due to nonspecific substrate binding by the antibody or streptavidin-fluorescein. Although these confocal binding studies utilized concentrations of phage (5×10⁹ pfu in 0.2-0.3 ml=1.7-2.5×10¹⁰ pfu/ml) that were on the same order of magnitude as those used in the phage binding studies (5×10¹⁰ pfu in 1 ml=5×10¹⁰ pfu/ml), relatively little binding was observed by the Graph5-01 or wildtype clones to SWNT paste in the phage binding studies as shown in FIG. 17. The differences in binding observed between these two experiments may be due to the manner in which the SWNT paste substrate was prepared and handled. The centrifugation of the wet, malleable SWNT paste used in the confocal experiments may have lead to trapping of both specific and nonspecific phage within the substrate, whereas the use of large dessicated SWNT aggregates in the phage binding studies may have prevented this. Wet paste was used in the confocal experiments to facilitate mounting under a coverslip, but future confocal binding experiments should utilize dessicated SWNT aggregates.

SWNT paste samples treated with peptides having sequences corresponding to the pIII inserts of the phage clones used above were also prepared but were not visualized.

Visualization of Phage on HOPG Using AFM

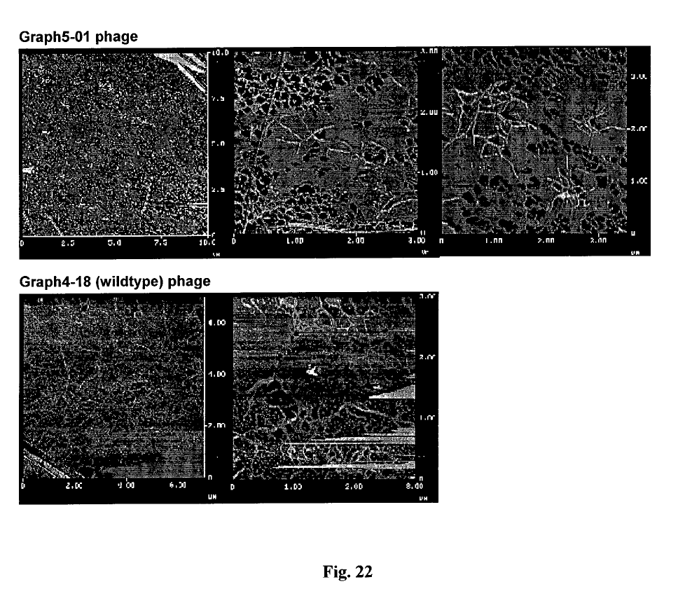

The binding of phage on carbon planchet and SWNT paste could not be analyzed using AFM due to the roughness of the substrate surfaces. Instead, HOPG was used and the results are shown in FIG. 22. Phage clone Graph5-01 (specific for carbon planchet) could be observed to bind to HOPG, whereas the wildtype clone was not readily observed on HOPG.

The phage binding studies and the visualization of peptides and phage binding to carbon planchets by confocal microscopy in this example consistently showed that the sequences N′-WWSWHPW-C′ (SEQ ID NO:238) and N′-HWSWWHP-C′ (SEQ ID NO:239) bound with greatest efficiencies to carbon planchet. Phage binding studies also revealed that the phage clone Hipco12R44-01 (N′-DMPRTTMSPPPR-C′) (SEQ ID NO:196) bound most efficiently to SWNT paste.

Little crossreactivity was observed in the phage binding studies and confocal experiments between the carbon planchet-specific phage clones and SWNT paste. Although the graphene structures present in the carbon planchets and SWNTs are theoretically very similar. It is possible that the walls of the SWNTs in the “raw” paste used in this studies contained contaminants and/or had been damaged by oxidation. To eliminate the possibility of the limited crossreactivity (i.e., high specificity) of the sequences due to the presence of possible contaminants, it may be desirable to use a purer nanotube source.

### EXAMPLE IV

### Applications of Biologic Materials with Affinities to Elemental Carbon-Containing Molecules

Examples illustrated below are illustrations of applications of the present invention, wherein SEQ ID NOS:1-245 may be used. In addition, examples may be applied using the methods and compositions of the present invention with other elemental carbon-containing molecules.

Separation Between Metallic and Semi-conducting CNT.

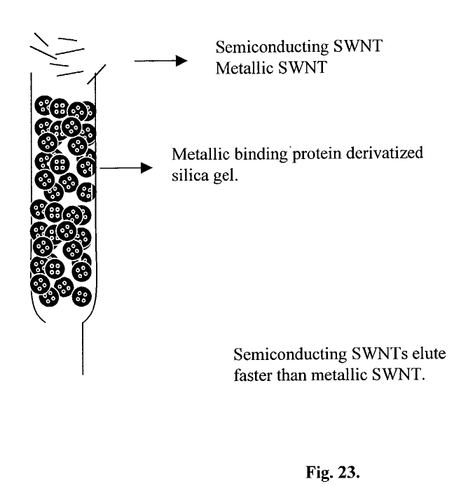

Current synthetic methods for producing single walled carbon nanotubes (SWNT) yield mixtures of metallic and semi-conducting SWNTs. In order to fabricate nanoscale electric devices, it is beneficial to separate the metallic SWNT and semi-conducting SWNT. Minute shape and symmetry differences between metallic and semi-conducting SWNT may be distinguished by the fast-evolved proteins obtained using the phage display or similar method. Based on the selected protein sequences from the phage display results, the negative column may be built to purify the mixture of metallic and semi-conducting SWNTs. If the mixture of metallic and semi-conducting SWNTs is passed through the negative column, the specific interaction between the peptides and one metallic or semi-conducting SWNTs cause the elution time difference. If metallic SWNTs binding peptides are applied to the negative column, the semi-conducting SWNTs elute faster than metallic SWNTs. Therefore, the one specific SWNT can be separated. A schematic diagram of SWNTs purifying negative column is shown in FIG. 23.

Alignment of Carbon Nanotubes

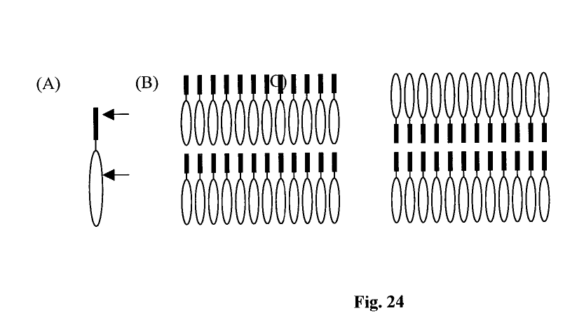

One of the greatest challenges in using carbon nanotubes as nanoscale devices is aligning the nanotubes in three-dimensional arrays. Although a chemical vapor deposition (CVD) method may produce unique aligned structure from the fabrication, a CVD method may also produce a mixture of metallic and semi-conducting SWNTs together. Because fabrication of the nano-electric devices is so precise, it is beneficial to separate the semi-conducting SWNTs from the mixture. The separation may be performed according to the method previously described. Although several approaches were used in this example such as LB-film method and meniscus force control, etc., these methods have produced only orientational aligned SWNT alignment. Both positionally and orientationally aligned SWNT 2D or 3D structures were built when phages having a specific binding property to SWNTs were used. SWNTs connected by phage as shown in FIG. 24, behave like di-block copolymers which have two rigid block connected by the peptide unit. It is expected that SWNT connected phage building blocks would produce microphase-separated lamellar like structure, with the resulting structure having aligned SWNT structures.

SWNT to P-N Junction SWNT by Peptide Binding

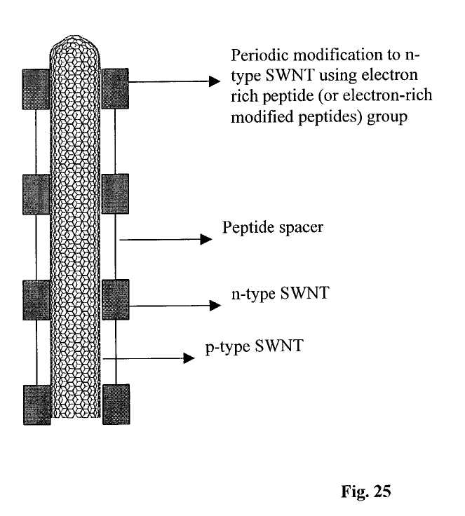

Without any chemical modification, semi-conducting SWNTs generally may have an intrinsic p-type electric property. Chemical modification with an electron-donating group may convert the p-type SWNT to n-type SWNT. Periodically bound peptides that generally have separate negatively and positively charged protein domains may cause the electronic properties of SWNTs. SWNTs that have periodic positively and negatively charged domains may be identical structures with P-N junction semiconductor structures. It is possible that the interconnection of these P-N junctions cause FET and higher architecture of complicated integrated circuit functions as NAND, NOR, AND, OR gates. A schematic diagram of n-type SWNT modification using SWNT binding peptides is shown in FIG. 25. These same modifications may be applied to multi-walled nanotubes and multi-walled nanotube pastes.

Solubility and Biocompatibility of Nanotubes

Low solubility in the solvent may block further application of SWNT. Generally, solubilization in water is essential for the biologic application of SWNT. Although wrapping polymers and surfactants were applied to solubilize the SWNT in this example, they must further be applied to biologic systems. It is believed that hydrophilic peptide groups conjugated with peptides that recognize the SWNT surfaces may solubilize the SWNT in water. In addition, removal of hydrophilic peptide groups may help SWNTs solubilize in non-polar solvents. These same modifications may be applied to multi-walled nanotubes and multi-walled nanotube pastes.

Wiring the Semi-Conducting SWNT

In accordance with the present invention, peptides recognizing SWNT's (metallic and semi conducting) may be wired together to form an integrated SWNT circuit and may serve as a functioning electric device. Similarly, the wiring technique may be applied to multi-walled nanotubes and other elemental carbon-containing molecules.

Biosensor

Biocompatible SWNTs may be utilized as a biosensor to detect minute chemical or physical changes in organisms. Conductivity of metallic SWNTs may generally be highly affected by the electron distribution around the SWNTs. As such, biologic interactions may be monitored by measuring the conductivity of SWNTs that are conjugated by two recognition moieties: one for SWNT and the other for the biologic targets. When the biologic target detecting-peptides bind with target molecules, the electron distribution in SWNTs may be affected by surrounding peptides. Binding and non-binding states of peptides may be monitored by electric signal and directly used as biosensors, such as antigen-antibody detection, glucose measurement in blood as well as others. Multi-walled nanotubes or other elemental carbon-containing molecules may also be used as biosensors using methods and compositions of the present invention.

Additionally, the peptide chain conformations that bind to SWNT are also affected by the pH, ionic strength, concentration of metal ion, and temperature changes. These environmental changes may also affect the electron distribution of SWNTs. All of these changes may be detected using SWNTs binding peptides.

8. Medication Release System

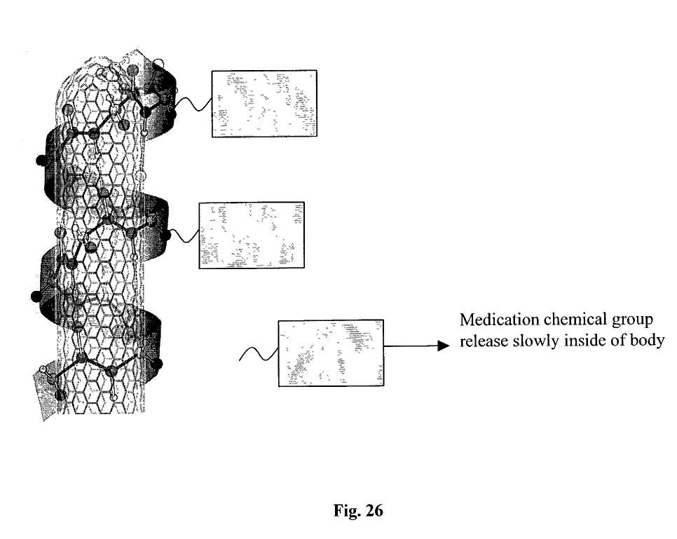

SWNTs may be used as robust scaffold to contain a drug. In addition, SWNTs may also be used to deliver a drug, especially if the SWNTs binding peptides are modified by the medications. For example, the medications connected by the peptides may slowly be released over time. Generally, these medications function similarly to patch-type medication delivery systems. A schematic diagram for the application of SWNT as a drug releasing system is shown in FIG. 26. In addition, the medication may be directly implanted into the disease-site such as for example, a tumor cell.

Other elemental carbon-containing molecules may also be used as pharmaceutical compositions of the present invention that release drugs, diagnostic markers, and/or medications to be used with methods and compositions of the present invention for preventive or prophylactic therapy, as treatment, for diagnosis, monitoring, and/or for screening (e.g., of drugs, symptoms, interactions, and/or effects).

Cancer Medication

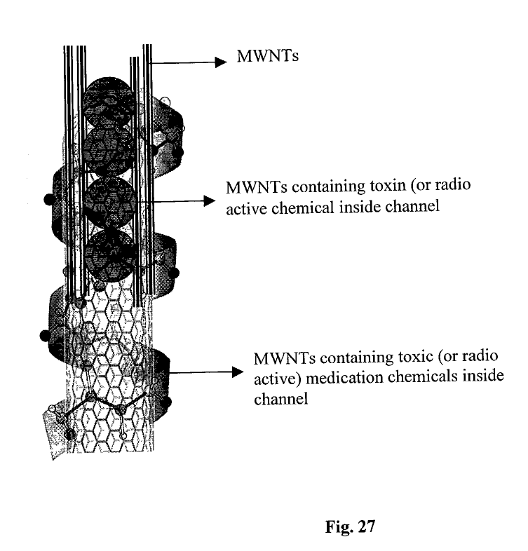

Biocompatible CNT may be used as radioactive or highly toxic medication delivery. In addition, multi-walled carbon nanotubes (MWNT) may be converted to biocompatible MWNT by peptides that have specific binding properties to MWNT. MWNTs generally contain at least about 3-4 nm of MWNT channel. This channel of MWNT may be filled by highly toxic or radioactive medications for special usage such as chemo-/radio-therapy. MWNTs that contain highly toxic or radioactive medication may then be directly implanted to the tumor cells or organism and thereafter, release the highly toxic or radioactive medication as desired. By changing the diameter of the inner channel, the releasing speed may be controlled. A schematic diagram for the application of SWNTs in cancer medication is shown in FIG. 27.

Other elemental carbon-containing molecules may also be used for the therapeutic delivery of agents as treatment tools or for monitoring disease progression (e.g., for cancer or other pathologic conditions).

The present invention may or may not include all the above-mentioned components. For example, biologic scaffolds of the present invention may be prepared in the absence of a substrate. In addition, the methods and compositions of the present invention may be applied for uses in fields such as optics, microelectronics, magnetics, and engineering. The applications include the synthesis of elemental carbon-containing materials, carbon nanutube alignment, creation of biologic semiconductors, junction conversion for single-walled nanotube paste, junction conversion for multi-walled nanotube paste, enhancing solubility and biologic compatability of single- and multi-walled nanotube paste, producing an integrated single- and multi-walled nanotube paste, biosensor production, release of pharmaceutical compositions, treatment of cancer, and combinations thereof.

While this invention has been described in reference to illustrative embodiments, this description is not intended to be construed in a limiting sense. Various modifications and combinations of the illustrative embodiments, as well as other embodiments of the invention will be apparent to persons skilled in the art upon reference to the description. It is therefore intended that the appended claims encompass any such modifications or embodiments.

What is claimed is:

1. A method for directed semiconductor formation comprising the steps of:
contacting a polymeric organic material that binds a predetermined face specificity semiconductor material with a first ion to create a semiconductor material precursor; and
adding a second ion to the semiconductor material precursor, wherein the polymeric organic material directs formation of the predetermined face specificity semiconductor material.

2. The method of claim 1, wherein the polymeric organic material is an amino acid oligomer.

3. The method of claim 1, wherein the polymeric organic material is an amino acid oligomer on the surface of a bacteriophage.

4. The method of claim 1, wherein the polymeric organic material is an amino acid oligomer displayed on the surface of bacteria.

5. The method of claim 1, wherein the polymeric organic material is an amino acid oligomer displayed on the surface of cell as a label.

6. The method of claim 1, wherein the polymeric organic material is a nucleic acid oligomer.

7. The method of claim 1, wherein the polymeric organic material is a combinatorial library.

8. The method of claim 1, wherein the polymeric organic material comprises amino acid polymers of between about 7 and 20 amino acids.

9. The method of claim 1, wherein the predetermined face specificity semiconductor material is polycrystalline.

10. The method of claim 1, wherein the predetermined face specificity semiconductor material is single crystalline.

11. The method of claim 1, wherein the predetermined face specificity semiconductor material comprises a Group II-IV semiconductor material.

12. The method of claim 1, wherein the polymeric organic material comprises a chimeric protein.

13. The method of claim 1, wherein the polymeric organic material comprises a chimeric protein and wherein the portion of the chimeric protein that binds the semiconductor material is on the surface of the chimeric protein.

14. The method of claim 1, wherein the polymeric organic material comprises a chimeric protein and wherein the portion of the chimeric protein that binds the semiconductor material comprises between about 7 and 20 amino acids.

15. The method of claim 1, wherein the polymeric organic material nucleates size constrained crystalline semiconductor materials.

16. The method of claim 1, wherein the polymeric organic material controls the crystallographic phase of nucleated nanoparticles of the semiconductor.

17. The method of claim 1, wherein the polymeric organic material controls the aspect ratio of the nanocrystals of the semiconductor.

18. The method of claim 1, wherein the polymeric organic material controls the dopant levels of the semiconductor nanocrystals formed.

19. A method for directed semiconductor formation comprising the steps of:
contacting a peptide that binds a predetermined face specificity semiconductor material with a first ion to create a semiconductor material precursor; and
adding a second ion to the semiconductor material precursor, wherein the peptide directs formation of the predetermined face specificity semiconductor material.

20. The method of claim 19, wherein the peptide is on the surface of a bacteriophage.

21. The method of claim 19, wherein the peptide is part of a combinatorial library.

22. The method of claim 19, wherein the peptide comprises between about 7 and 20 amino acids.

23. The method of claim 19, wherein the predetermined face specificity semiconductor material is polycrystalline.

24. The method of claim 19, wherein the predetermined face specificity semiconductor material is single crystalline.

25. The method of claim 19, wherein the predetermined face specificity semiconductor material comprises a Group II-VI semiconductor material.

26. The method of claim 19, wherein the polymeric organic material is displayed on the surface of bacteria.

27. The method of claim 19, wherein the polymeric organic material is displayed on the surface of cell as a label.

28. The method of claim 19, wherein the peptide comprises a chimeric protein.

29. The method of claim 19, wherein the peptide comprises a chimeric protein and wherein the peptide portion of the chimeric protein that binds the semiconductor material is on the surface of the chimeric protein.

30. The method of claim 19, wherein the peptide comprises a chimeric protein and wherein the portion of the chimeric protein that binds the semiconductor material comprises between about 7 and 20 amino acids.

31. The method of claim 19, wherein the peptide nucleates size constrained crystalline semiconductor materials.

32. The method of claim 19, wherein the peptide controls the crystallographic phase of nucleated nanoparticles of the semiconductor.

33. The method of claim 19, wherein the peptide is selected from a 12 mer linear library.

34. The method of claim 19, wherein the peptide is selected from a 7 mer constrained library.

35. A method for nucleating semiconductor material comprising the steps of:
selecting a peptide that binds to a predetermined face specificity material;
preparing a portion of a gold surface that has been altered to have the peptide attached to the surface;
contacting the gold surface-peptide complex with a first ion needed for semiconductor crystal precursor formation; and
adding a second ions needed for semiconductor crystal formation.

36. The method of claim 35, wherein the peptide is selected from a constrained library.

37. The method of claim 35, wherein the gold-surface is prepared by forming a self-assembled monolayer with 2-mercaptoethylamine on the gold substrate.

38. The method of claim 35, wherein the predetermined face specificity semiconductor material comprises a Group II-VI semiconductor material.

39. The method of claim 35, wherein the semiconductor material is zinc sulfide and the solutions are zinc chloride and sodium sulfide.

40. The method of claim 35, wherein the semiconductor material is cadmium sulfide and the solutions are cadmium chloride and sodium sulfide.

41. The method of claim 35, wherein the peptide is selected by combinatorial library screening.

42. A method of constructing nanowires comprising the steps of:
selecting peptides that bind a predetermined face specificity semiconductor material; and
expressing the peptides as a fusion protein with a protein that is capable of self-assembly;
then interact fused with semiconductor precusors to direct formation of semiconductor nanocrystals.

43. The method of claim 42, wherein the peptides selected are expressed in high copy number.

44. The method of claim 42, wherein the self-assembled protein is on the surface of a bacteriophage.

45. The method of claim 42, wherein the polymeric organic material is displayed on the surface of bacteria.

46. The method of claim 42, wherein the polymeric organic material is displayed on the surface of cell as a label.

47. The method of claim 42, wherein the self-assembled protein comprises a portion of the major coat protein of M1 bacteriophage.

48. The method of claim 42, wherein the self-assembled protein comprises a portion of the p8 major coat protein of M1 bacteriophage.

49. A semiconductor made using the process of claim 1.

50. A semiconductor material made using the process of claim 15.

51. A nanowire made using the process of claim 35.

52. A biologic scaffold comprising:
a substrate capable of binding one or more biologic materials;
one or more biologic materials attached to the substrate; and
one or more elemental carbon-containing molecules attached to one or more biologic materials.

53. The biologic scaffold of claim 52, wherein the substrate is selected from the group consisting of silicon, Langmuir-Bodgett films, functionalized glass, germanium, ceramic, silicon, a semiconductor material, PTFE, carbon, polycarbonate, mica, mylar, plastic, quartz, polystyrene, gallium arsenide, gold, silver, metal, metal alloy, fabric, tissue, cell, organ, protein, antibody, and combinations thereof.

54. The biologic scaffold of claim 52, wherein the biologic material is selected from the group consisting of virus, bacteriophage, bacteria, peptide, protein, amino acid, steroid, drug, chromophore, antibody, enzyme, single-stranded or double-stranded nucleic acid, nucleic acid polymer, and any chemical modifications thereof.

55. The biologic scaffold of claim 52, wherein the biologic material is identified by a combinatorial library screening.

56. The biologic scaffold of claim 52, wherein the biologic material is an amino acid oligomer present on the surface of a bacteriophage.

57. The biologic scaffold of claim 52, wherein the biologic material is an amino acid oligomer displayed on the surface of bacteria.

58. The biologic scaffold of claim 52, wherein the biologic material is an amino acid oligomer between 7 and 20 amino acids long.

59. The biologic scaffold of claim 52, wherein the biologic material is a peptide on the surface of a bacteriophage.

60. The biologic scaffold of claim 59, wherein the biologic material is a peptide selected from the group consisting of SEQ ID NO.:105-245.

61. The biologic scaffold of claim 52, wherein the elemental carbon-containing molecule recognizes a peptide selected from the group consisting of SEQ ID NO.:105-245.

62. The biologic scaffold of claim 52, wherein the elemental carbon-containing molecule is selected from the group consisting of carbon₆₀, carbon planchet, highly ordered pyrolytic graphite, single-walled nanotube paste, single-walled nanotube, multi-walled nanotube, multi-walled nanotube paste, diamond, graphite, activated carbon, carbon black, industrial carbon, charcoal, coke, steel, carbon cycle, and combinations thereof.

63. The biologic scaffold of claim 52, wherein the substrate is absent from the biologic scaffold.

64. The biologic scaffold of claim 52, wherein the biologic scaffold is used for applications selected from the group consisting of synthesis of elemental carbon-containing materials, carbon nanutube alignment, creation of biologic semiconductors, junction conversion for single-walled nanotube paste, junction conversion for multi-walled nanotube paste, enhancing solubility and biologic compatability of single- and multi-walled nanotube paste, producing an integrated single- and multi-walled nanotube paste, biosensor production, release of pharmaceutical compositions, treatment of cancer, and combinations thereof.

65. A biologic scaffold comprising:
a substrate capable of binding one or more biologic materials;
a biologic material attached to the substrate and an organic polymer attached to the biologic material; and
one or more elemental carbon-containing molecules attached to the organic polymer.

66. The biologic scaffold of claim 65, wherein the substrate is selected from the group consisting of silicon, Langmuir-Bodgett films, functionalized glass, germanium, ceramic, silicon, a semiconductor material, PTFE, carbon, polycarbonate, mica, mylar, plastic, quartz, polystyrene, gallium arsenide, gold, silver, metal, metal alloy, fabric, tissue, cell, organ, protein, antibody, and combinations thereof.

67. The biologic scaffold of claim 65, wherein the biologic material is selected from the group consisting of virus, bacteriophage, bacteria, peptide, protein, amino acid, steroid, drug, chromophore, antibody, enzyme, single-stranded or double-stranded nucleic acid, nucleic acid polymer, and any chemical modifications thereof.

68. The biologic scaffold of claim 65, wherein the biologic material and organic polymer are the same.

69. The biologic scaffold of claim 65, wherein the organic polymer is a protein, antibody, peptide, nucleic acid, chimeric molecule, drug, label, other carbon-containing organic materials known to exist in eukaryotic organisms, and derivatives or analogs of biologic polymers that contain one or more biologic monomers in combinations with synthetic monomers that mimic those found naturally.

70. The biologic scaffold of claim 65, wherein the organic polymer is identified by a combinatorial library screening.

71. The biologic scaffold of claim 65, wherein the organic polymer is an amino acid oligomer between 7 and 20 amino acids long.

72. The biologic scaffold of claim 65, wherein the organic polymer is a peptide that recognizes a select portion of the biologic material

73. The biologic scaffold of claim 65, wherein the second biologic material is a peptide selected from the group consisting of SEQ ID NO.: 105-245.

74. The biologic scaffold of claim 65, wherein the elemental carbon-containing molecule recognizes a peptide selected from the group consisting of SEQ ID NO.:105-245.

75. The biologic scaffold of claim 65, wherein the elemental carbon-containing molecule is selected from the group consisting of carbon₆₀, carbon planchet, highly ordered pyrolytic graphite, single-walled nanotube paste, single-walled nanotube, multi-walled nanotube, multi-walled nanotube paste, diamond, graphite, activated carbon, carbon black, industrial carbon, charcoal, coke, steel, carbon cycle, and combinations thereof.

76. The biologic scaffold of claim 65, wherein the biologic scaffold is used for applications selected from the group consisting of synthesis of elemental carbon-containing materials, carbon nanutube alignment, creation of biologic semiconductors, junction conversion for single-walled nanotube paste, junction conversion for multi-walled nanotube paste, enhancing solubility and biologic compatability of single- and multi-walled nanotube paste, producing an integrated single- and multi-walled nanotube paste, biosensor production, release of pharmaceutical compositions, treatment of cancer, and combinations thereof.

77. The biologic scaffold of claim 65, wherein the substrate and the biologic material are the same.

78. A biologic scaffold comprising:
a substrate capable of binding one or more bacteriophages;
one or more bacteriophages attached to the substrate;
one or more peptides that recognize a portion of the bacteriophage; and
one or more elemental carbon-containing molecules that recognize the peptide.

79. The biologic scaffold of claim 78, wherein the substrate is silicon, Langmuir-Bodgett films, functionalized glass, germanium, ceramic, silicon, a semiconductor material, PTFE, carbon, polycarbonate, mica, mylar, plastic, quartz, polystyrene, gallium arsenide, gold, silver, metal, metal alloy, fabric, tissue, cell, organ, protein, antibody, and combinations thereof.

80. The biologic scaffold of claim 78, wherein the peptide is selected from the group consisting of SEQ ID NO.:105-245.

81. The biologic scaffold of claim 78, wherein the elemental carbon-containing molecule is selected from the group consisting of carbon₆₀, carbon planchet, highly ordered pyrolytic graphite, single-walled nanotube paste, single-walled nanotube, multi-walled nanotube, multi-walled nanotube paste, diamond, graphite, activated carbon, carbon black, industrial carbon, charcoal, coke, steel, carbon cycle, and combinations thereof.

82. The biologic scaffold of claim 78, wherein the peptide is selected from the group consisting of drug, antibody, chromophore, light-emitting label, light absorbing label, and organic polymer.

83. The biologic scaffold of claim 78, wherein the substrate is absent.

84. A method of making a biologic scaffold comprising:
providing a substrate capable of binding one or more biologic materials;
attaching one or more biologic materials to the substrate; and
contacting one or more elemental carbon-containing molecules with the biologic material to form a biologic scaffold.

85. The method of claim 84, wherein the substrate is selected from the group consisting of silicon, Langmuir-Bodgett films, functionalized glass, germanium, ceramic, silicon, a semiconductor material, PTFE, carbon, polycarbonate, mica, mylar, plastic, quartz, polystyrene, gallium arsenide, gold, silver, metal, metal alloy, fabric, tissue, cell, organ, protein, antibody, and combinations thereof.

86. The method of claim 84, wherein the biologic material is selected from the group consisting of virus, bacteriophage, bacteria, peptide, protein, amino acid, steroid, drug, chromophore, label, antibody, enzyme, single-stranded or double-stranded nucleic acid, nucleic acid polymer, chimeric molecule, drug, any other carbon-containing materials known to exist in eukaryotic organisms, and derivatives or analogs of biologic polymers that contain one or more biologic monomers in combination with synthetic monomers that mimic those found naturally.

87. The method of claim 84, wherein the biologic material is identified by combinatorial library screening.

88. The method of claim 84, wherein the biologic material is an amino acid oligomer on the surface of a bacteriophage.

89. The method of claim 84, wherein the biologic material is a peptide displayed on the surface of bacteria.

90. The method of claim 88, wherein the amino acid oligomer is between 7 and 20 amino acids long.

91. The method of claim 89, wherein the peptide is selected from the group consisting of SEQ ID NO.:105-245.

92. The method of claim 89, wherein the peptide is selected from the group consisting of drug, antibody, chromophore, light-emitting label, light absorbing label, and organic polymer.

93. The method of claim 84, wherein the elemental carbon-containing molecule recognizes a peptide selected from the group consisting of SEQ ID NO.:105-245.

94. The method of claim 84, wherein the elemental carbon-containing molecule is selected from the group consisting of carbon₆₀, carbon planchet, highly ordered pyrolytic graphite, single-walled nanotube paste, single-walled nanotube, multi-walled nanotube, multi-walled nanotube paste, diamond, graphite, activated carbon, carbon black, industrial carbon, charcoal, coke, steel, carbon cycle, and combinations thereof.

95. The method of claim 84, wherein providing a substrate capable of binding one or more biologic materials and attaching one or more biologic materials to the substrate are not required to make the biologic scaffold.

96. A molecule comprising:
an organic polymer, wherein the organic polymer selectively recognizes an elemental carbon-containing molecule.

97. The molecule of claim 96, wherein the molecule is used for applications selected from the group consisting of synthesis of elemental carbon-containing materials, carbon nanutube alignment, creation of biologic semiconductors, junction conversion for single-walled nanotube paste, junction conversion for multi-walled nanotube paste, enhancing solubility and biologic compatability of single- and multi-walled nanotube paste, producing an integrated single- and multi-walled nanotube paste, biosensor production, release of pharmaceutical compositions, treatment of cancer, and combinations thereof.

98. The molecule of claim 96, wherein the organic polymer is a nucleic acid oligomer.

99. The molecule of claim 96, wherein the organic polymer is selected by a combinatorial library screening.

100. The molecule of claim 96, wherein the organic polymer is an amino acid oligomer on the surface of a bacteriophage.

101. The molecule of claim 100, wherein the amino acid oligomer is displayed on the surface of bacteria.

102. The molecule of claim 100, wherein the amino acid oligomer is between 7 and 15 amino acids long.

103. The molecule of claim 96, wherein the organic polymer is a peptide on the surface of a bacteriophage.

104. The molecule of claim 103, wherein the peptide is selected from the group consisting of SEQ ID NO.:105-245.

105. The molecule of claim 96, wherein the elemental carbon-containing molecule recognizes a peptide selected from the group consisting of SEQ ID NO.:105-245.

106. The molecule of claim 96, wherein the elemental carbon-containing molecule is selected from the group consisting of carbon₆₀, carbon planchet, highly ordered pyrolytic graphite, single-walled nanotube paste, single-walled nanotube, multi-walled nanotube, multi-walled nanotube paste, diamond, graphite, activated carbon, carbon black, industrial carbon, charcoal, coke, steel, carbon cycle, and combinations thereof.

107. An integrated circuit derived from the biologic scaffold of claim 52.

108. A biosensor derived from the biologic scaffold of claim 52.

109. A drug delivery system using the biologic scaffold of claim 52.

110. A pharmaceutical composition using a pharmaceutically effective amount of the molecule of claim 96.

111. A treatment for cancer using the biologic scaffold of claim 52.

112. A method for separating metallic and semi-conducting nanotubes comprising the steps of:
obtaining protein sequences using a combinatorial library screening that distinguishes metallic and semi-conducting nanotubes;
contacting a mixture of metallic and semi-conducting nanotubes with the obtained protein sequences; and p1 separating the semi-conducting nanotube from the metallic nanotube

113. The method of claim 112, wherein metallic and semi-conducting nanotubes are selected from the group consisting of single-walled nanotubes and multi-walled nanotubes.


SEQUENCE LISTING (245 sequences)

SEQ ID NO: 1 - protein, 12 aa, artificial sequence
  artifical peptide with peptide binding sequence retrieved from phage biopanning
AMAGTTSDPSTV

SEQ ID NO: 2 - protein, 12 aa, artificial sequence
  peptide with peptide binding sequence retrieved from phage biopanning
AASPTQSMSQAP

SEQ ID NO: 3 - protein, 12 aa, artificial sequence
  peptide with peptide binding sequence retrieved from phage biopanning
HTHTNNDSPNQA

SEQ ID NO: 4 - protein, 12 aa, artificial sequence
  peptide with peptide binding sequence retrieved from phage biopanning
DTQGFHSRSSSA

SEQ ID NO: 5 - protein, 12 aa, artificial sequence
  peptide with peptide binding sequence retrieved from phage biopanning
TSSSALQPAHAW

SEQ ID NO: 6 - protein, 12 aa, artificial sequence
  peptide with peptide binding sequence retrieved from phage biopanning
SESSPISLDYRA

SEQ ID NO: 7 - protein, 12 aa, artificial sequence
  peptide with peptide binding sequence retrieved from phage biopanning
STHNYQIPRPPT

SEQ ID NO: 8 - protein, 12 aa, artificial sequence
  peptide with peptide binding sequence retrieved from phage biopanning
HPFSNEPLQLSS

SEQ ID NO: 9 - protein, 12 aa, artificial sequence
  peptide with peptide binding sequence retrieved from phage biopanning
GTLANQQIFLSS

SEQ ID NO: 10 - protein, 12 aa, artificial sequence
  peptide with peptide binding sequence retrieved from phage biopanning
HGNPLPMTPFPG

SEQ ID NO: 11 - protein, 12 aa, artificial sequence
  peptide with peptide binding sequence retrieved from phage biopanning
RLELAIPLQGSG

SEQ ID NO: 12 - protein, 9 aa, artificial sequence
  peptide with peptide binding sequence retrieved from phage biopanning
CHASNRLSC

SEQ ID NO: 13 - protein, 12 aa, artificial sequence
  peptide with peptide binding sequence retrieved from phage biopanning
SMDRSDMTMRLP

SEQ ID NO: 14 - protein, 12 aa, artificial sequence
  peptide with peptide binding sequence retrieved from phage biopanning
GTFTPRPTPIYP

SEQ ID NO: 15 - protein, 12 aa, artificial sequence
  peptide with peptide binding sequence retrieved from phage biopanning
QMSENLTSQIES

SEQ ID NO: 16 - protein, 12 aa, artificial sequence
  peptide with peptide binding sequence retrieved from phage biopanning
DMLARLRATAGP

SEQ ID NO: 17 - protein, 12 aa, artificial sequence
  peptide with peptide binding sequence retrieved from phage biopanning
SQTWLLMSPVAT

SEQ ID NO: 18 - protein, 12 aa, artificial sequence
  peptide with peptide binding sequence retrieved from phage biopanning
ASPDQQVGPLYV

SEQ ID NO: 19 - protein, 12 aa, artificial sequence
  peptide with peptide binding sequence retrieved from phage biopanning
LTWSPLQTVARF

SEQ ID NO: 20 - protein, 12 aa, artificial sequence
  peptide with peptide binding sequence retrieved from phage biopanning
QISAHQMPSRPI

SEQ ID NO: 21 - protein, 12 aa, artificial sequence
  peptide with peptide binding sequence retrieved from phage biopanning
SMKYNLIVDSPY

SEQ ID NO: 22 - protein, 12 aa, artificial sequence
  peptide with peptide binding sequence retrieved from phage biopanning
QMPIRNQLAWPM

SEQ ID NO: 23 - protein, 12 aa, artificial sequence
  peptide with peptide binding sequence retrieved from phage biopanning
TQNLEIREPLTP

SEQ ID NO: 24 - protein, 12 aa, artificial sequence
  peptide with peptide binding sequence retrieved from phage biopanning
YPMSPSPYPYQL

SEQ ID NO: 25 - protein, 12 aa, artificial sequence
  peptide with peptide binding sequence retrieved from phage biopanning
SFMIQPTPLPPS

SEQ ID NO: 26 - protein, 12 aa, artificial sequence
  peptide with peptide binding sequence retrieved from phage biopanning
GLAPHIHSLNEA

SEQ ID NO: 27 - protein, 12 aa, artificial sequence
  peptide with peptide binding sequence retrieved from phage biopanning
MQFPVTPYLNAS

SEQ ID NO: 28 - protein, 12 aa, artificial sequence
  peptide with peptide binding sequence retrieved from phage biopanning
SPGDSLKKLAAS

SEQ ID NO: 29 - protein, 12 aa, artificial sequence
  peptide with peptide binding sequence retrieved from phage biopanning
GYHMQTLPGPVA

SEQ ID NO: 30 - protein, 12 aa, artificial sequence
  peptide with peptide binding sequence retrieved from phage biopanning
SLTPLTTSHLRS

SEQ ID NO: 31 - protein, 12 aa, artificial sequence
  peptide with peptide binding sequence retrieved from phage biopanning
TLTNGPLRPFTG

SEQ ID NO: 32 - protein, 12 aa, artificial sequence
  peptide with peptide binding sequence retrieved from phage biopanning
LNTPKPFTLGQN

SEQ ID NO: 33 - protein, 9 aa, artificial sequence
  peptide with peptide binding sequence retrieved from phage biopanning
CDLQNYKAC

SEQ ID NO: 34 - protein, 9 aa, artificial sequence
  peptide with peptide binding sequence retrieved from phage biopanning
CRHPHTRLC

SEQ ID NO: 35 - protein, 9 aa, artificial sequence
  peptide with peptide binding sequence retrieved from phage biopanning
CANLKPKAC

SEQ ID NO: 36 - protein, 9 aa, artificial sequence
  peptide with peptide binding sequence retrieved from phage biopanning
CYINPPKVC

SEQ ID NO: 37 - protein, 9 aa, artificial sequence
  peptide with peptide binding sequence retrieved from phage biopanning
CNNKVPVLC

SEQ ID NO: 38 - protein, 9 aa, artificial sequence
  peptide with peptide binding sequence retrieved from phage biopanning
CHASKTPLC

SEQ ID NO: 39 - protein, 9 aa, artificial sequence
  peptide with peptide binding sequence retrieved from phage biopanning
CASQLYPAC

SEQ ID NO: 40 - protein, 9 aa, artificial sequence
  peptide with peptide binding sequence retrieved from phage biopanning
CNMTQYPAC

SEQ ID NO: 41 - protein, 9 aa, artificial sequence
  peptide with peptide binding sequence retrieved from phage biopanning
CFAPSGPAC

SEQ ID NO: 42 - protein, 9 aa, artificial sequence
  peptide with peptide binding sequence retrieved from phage biopanning
CPVWIQAPC

SEQ ID NO: 43 - protein, 9 aa, artificial sequence
  peptide with peptide binding sequence retrieved from phage biopanning
CQVAVNPLC

SEQ ID NO: 44 - protein, 9 aa, artificial sequence
  peptide with peptide binding sequence retrieved from phage biopanning
CQPEAMPAC

SEQ ID NO: 45 - protein, 9 aa, artificial sequence
  peptide with peptide binding sequence retrieved from phage biopanning
CHPTMPLAC

SEQ ID NO: 46 - protein, 9 aa, artificial sequence
  peptide with peptide binding sequence retrieved from phage biopanning
CPPFAAPIC

SEQ ID NO: 47 - protein, 9 aa, artificial sequence
  peptide with peptide binding sequence retrieved from phage biopanning
CNKHQPMHC

SEQ ID NO: 48 - protein, 9 aa, artificial sequence
  peptide with peptide binding sequence retrieved from phage biopanning
CFPMRSNQC

SEQ ID NO: 49 - protein, 9 aa, artificial sequence
  peptide with peptide binding sequence retrieved from phage biopanning
CQSMPHNRC

SEQ ID NO: 50 - protein, 9 aa, artificial sequence
  peptide with peptide binding sequence retrieved from phage biopanning
CNNPMHQNC

SEQ ID NO: 51 - protein, 9 aa, artificial sequence
  peptide with peptide binding sequence retrieved from phage biopanning
CHMAPRWQC

SEQ ID NO: 52 - protein, 9 aa, artificial sequence
  peptide with peptide binding sequence retrieved from phage biopanning
HVHIHSRPM

SEQ ID NO: 53 - protein, 9 aa, artificial sequence
  peptide with peptide binding sequence retrieved from phage biopanning
LPNMHPLPL

SEQ ID NO: 54 - protein, 9 aa, artificial sequence
  peptide with peptide binding sequence retrieved from phage biopanning
LPLRLPPMP

SEQ ID NO: 55 - protein, 9 aa, artificial sequence
  peptide with peptide binding sequence retrieved from phage biopanning
HSMIGTPTT

SEQ ID NO: 56 - protein, 9 aa, artificial sequence
  peptide with peptide binding sequence retrieved from phage biopanning
SVSVGMKPS

SEQ ID NO: 57 - protein, 9 aa, artificial sequence
  peptide with peptide binding sequence retrieved from phage biopanning
LDASFMQDW

SEQ ID NO: 58 - protein, 9 aa, artificial sequence
  peptide with peptide binding sequence retrieved from phage biopanning
TPPSYQMAM

SEQ ID NO: 59 - protein, 9 aa, artificial sequence
  peptide with peptide binding sequence retrieved from phage biopanning
YPQLVSMST

SEQ ID NO: 60 - protein, 9 aa, artificial sequence
  peptide with peptide binding sequence retrieved from phage biopanning
GYSTINMYS

SEQ ID NO: 61 - protein, 9 aa, artificial sequence
  peptide with peptide binding sequence retrieved from phage biopanning
DRMLLPFNL

SEQ ID NO: 62 - protein, 9 aa, artificial sequence
  peptide with peptide binding sequence retrieved from phage biopanning
IPMTPSYDS

SEQ ID NO: 63 - protein, 9 aa, artificial sequence
  peptide with peptide binding sequence retrieved from phage biopanning
MYSPRPPAL

SEQ ID NO: 64 - protein, 9 aa, artificial sequence
  peptide with peptide binding sequence retrieved from phage biopanning
QPTTDLMAH

SEQ ID NO: 65 - protein, 9 aa, artificial sequence
  peptide with peptide binding sequence retrieved from phage biopanning
ATHVQMAWA

SEQ ID NO: 66 - protein, 9 aa, artificial sequence
  peptide with peptide binding sequence retrieved from phage biopanning
SMHATLTPM

SEQ ID NO: 67 - protein, 9 aa, artificial sequence
  peptide with peptide binding sequence retrieved from phage biopanning
SGPAHGMFA

SEQ ID NO: 68 - protein, 9 aa, artificial sequence
  peptide with peptide binding sequence retrieved from phage biopanning
IANRPYSAQ

SEQ ID NO: 69 - protein, 7 aa, artificial sequence
  peptide with peptide binding sequence retrieved from phage biopanning
VMTQPTR

SEQ ID NO: 70 - protein, 7 aa, artificial sequence
  peptide with peptide binding sequence retrieved from phage biopanning
HMRPLSI

SEQ ID NO: 71 - protein, 12 aa, artificial sequence
  peptide with peptide binding sequence retrieved from phage biopanning
LTRSPLHVDQRR

SEQ ID NO: 72 - protein, 12 aa, artificial sequence
  peptide with peptide binding sequence retrieved from phage biopanning
VISNHAESSRRL

SEQ ID NO: 73 - protein, 7 aa, artificial sequence
  peptide with peptide binding sequence retrieved from phage biopanning
HTHIPNQ

SEQ ID NO: 74 - protein, 7 aa, artificial sequence
  peptide with peptide binding sequence retrieved from phage biopanning
LAPVSPP

SEQ ID NO: 75 - protein, 9 aa, artificial sequence
  peptide with peptide binding sequence retrieved from phage biopanning
CMTAGKNTC

SEQ ID NO: 76 - protein, 9 aa, artificial sequence
  peptide with peptide binding sequence retrieved from phage biopanning
CQTLWRNSC

SEQ ID NO: 77 - protein, 9 aa, artificial sequence
  peptide with peptide binding sequence retrieved from phage biopanning
CTSVHTNTC

SEQ ID NO: 78 - protein, 9 aa, artificial sequence
  peptide with peptide binding sequence retrieved from phage biopanning
CPSLAMNSC

SEQ ID NO: 79 - protein, 9 aa, artificial sequence
  peptide with peptide binding sequence retrieved from phage biopanning
CSNNTVHAC

SEQ ID NO: 80 - protein, 9 aa, artificial sequence
  peptide with peptide binding sequence retrieved from phage biopanning
CLPAQGHVC

SEQ ID NO: 81 - protein, 9 aa, artificial sequence
  peptide with peptide binding sequence retrieved from phage biopanning
CLPAQVHVC

SEQ ID NO: 82 - protein, 9 aa, artificial sequence
  peptide with peptide binding sequence retrieved from phage biopanning
CPPKNVRLC

SEQ ID NO: 83 - protein, 9 aa, artificial sequence
  peptide with peptide binding sequence retrieved from phage biopanning
CPHINAHAC

SEQ ID NO: 84 - protein, 9 aa, artificial sequence
  peptide with peptide binding sequence retrieved from phage biopanning
CIVNLARAC

SEQ ID NO: 85 - protein, 12 aa, artificial sequence
  peptide with peptide binding sequence retrieved from phage biopanning
TMGFTAPRFPHY

SEQ ID NO: 86 - protein, 12 aa, artificial sequence
  peptide with peptide binding sequence retrieved from phage biopanning
ATQSYVRHPSLG

SEQ ID NO: 87 - protein, 12 aa, artificial sequence
  peptide with peptide binding sequence retrieved from phage biopanning
TSTTQGALAYLF

SEQ ID NO: 88 - protein, 12 aa, artificial sequence
  peptide with peptide binding sequence retrieved from phage biopanning
DPPWSAIVRHRD

SEQ ID NO: 89 - protein, 12 aa, artificial sequence
  peptide with peptide binding sequence retrieved from phage biopanning
FDNKPFLRVASE

SEQ ID NO: 90 - protein, 12 aa, artificial sequence
  peptide with peptide binding sequence retrieved from phage biopanning
HQSHTQQNKRHL

SEQ ID NO: 91 - protein, 12 aa, artificial sequence
  peptide with peptide binding sequence retrieved from phage biopanning
TSTTQGALAYLF

SEQ ID NO: 92 - protein, 12 aa, artificial sequence
  peptide with peptide binding sequence retrieved from phage biopanning
KTPIHTSAWEFQ

SEQ ID NO: 93 - protein, 12 aa, artificial sequence
  peptide with peptide binding sequence retrieved from phage biopanning
DLFHLKPVSNEK

SEQ ID NO: 94 - protein, 12 aa, artificial sequence
  peptide with peptide binding sequence retrieved from phage biopanning
KPFWTSSPDVMT

SEQ ID NO: 95 - protein, 12 aa, artificial sequence
  peptide with peptide binding sequence retrieved from phage biopanning
PWAATSKPPYSS

SEQ ID NO: 96 - protein, 9 aa, artificial sequence
  peptide with peptide binding sequence retrieved from phage biopanning
CQNPMQTFC

SEQ ID NO: 97 - protein, 9 aa, artificial sequence
  peptide with peptide binding sequence retrieved from phage biopanning
CNQLSTRPC

SEQ ID NO: 98 - protein, 9 aa, artificial sequence
  peptide with peptide binding sequence retrieved from phage biopanning
CLQNRQSQC

SEQ ID NO: 99 - protein, 9 aa, artificial sequence
  peptide with peptide binding sequence retrieved from phage biopanning
CQLQRQWNC

SEQ ID NO: 100 - protein, 9 aa, artificial sequence
  peptide with peptide binding sequence retrieved from phage biopanning
CQVNSAHQC

SEQ ID NO: 101 - protein, 9 aa, artificial sequence
  peptide with peptide binding sequence retrieved from phage biopanning
CFPMRSNQC

SEQ ID NO: 102 - protein, 9 aa, artificial sequence
  peptide with peptide binding sequence retrieved from phage biopanning
CPPQPNRQC

SEQ ID NO: 103 - protein, 9 aa, artificial sequence
  peptide with peptide binding sequence retrieved from phage biopanning
CQMPMQHNC

SEQ ID NO: 104 - protein, 9 aa, artificial sequence
  peptide with peptide binding sequence retrieved from phage biopanning
CANVAQRNC

SEQ ID NO: 105 - protein, 9 aa, artificial sequence
  peptide with peptide binding sequence retrieved from phage biopanning
CNNKQLYYC

SEQ ID NO: 106 - protein, 9 aa, artificial sequence
  peptide with peptide binding sequence retrieved from phage biopanning
CQTAWIGQC

SEQ ID NO: 107 - protein, 9 aa, artificial sequence
  peptide with peptide binding sequence retrieved from phage biopanning
CQSANKLTC

SEQ ID NO: 108 - protein, 9 aa, artificial sequence
  peptide with peptide binding sequence retrieved from phage biopanning
CIPYTMAMC

SEQ ID NO: 109 - protein, 9 aa, artificial sequence
  peptide with peptide binding sequence retrieved from phage biopanning
CLPSYHNNC

SEQ ID NO: 110 - protein, 9 aa, artificial sequence
  peptide with peptide binding sequence retrieved from phage biopanning
CVSVAHKDC

SEQ ID NO: 111 - protein, 9 aa, artificial sequence
  peptide with peptide binding sequence retrieved from phage biopanning
CEVTTLYRC

SEQ ID NO: 112 - protein, 9 aa, artificial sequence
  peptide with peptide binding sequence retrieved from phage biopanning
CELTAFPAC

SEQ ID NO: 113 - protein, 9 aa, artificial sequence
  peptide with peptide binding sequence retrieved from phage biopanning
CTLASPHQC

SEQ ID NO: 114 - protein, 9 aa, artificial sequence
  peptide with peptide binding sequence retrieved from phage biopanning
CPLTGGPTC

SEQ ID NO: 115 - protein, 9 aa, artificial sequence
  peptide with peptide binding sequence retrieved from phage biopanning
CWWSWHPWC

SEQ ID NO: 116 - protein, 9 aa, artificial sequence
  peptide with peptide binding sequence retrieved from phage biopanning
CQKSGVHLC

SEQ ID NO: 117 - protein, 9 aa, artificial sequence
  peptide with peptide binding sequence retrieved from phage biopanning
CLFNALIRC

SEQ ID NO: 118 - protein, 9 aa, artificial sequence
  peptide with peptide binding sequence retrieved from phage biopanning
CVMWTSHSC

SEQ ID NO: 119 - protein, 9 aa, artificial sequence
  peptide with peptide binding sequence retrieved from phage biopanning
CVSRWRASC

SEQ ID NO: 120 - protein, 9 aa, artificial sequence
  peptide with peptide binding sequence retrieved from phage biopanning
CSSWEPKSC

SEQ ID NO: 121 - protein, 9 aa, artificial sequence
  peptide with peptide binding sequence retrieved from phage biopanning
CTLTGPFAC

SEQ ID NO: 122 - protein, 9 aa, artificial sequence
  peptide with peptide binding sequence retrieved from phage biopanning
CPPVLGNLC

SEQ ID NO: 123 - protein, 9 aa, artificial sequence
  peptide with peptide binding sequence retrieved from phage biopanning
CPHAPSGPC

SEQ ID NO: 124 - protein, 9 aa, artificial sequence
  peptide with peptide binding sequence retrieved from phage biopanning
CPLHKNGKC

SEQ ID NO: 125 - protein, 9 aa, artificial sequence
  peptide with peptide binding sequence retrieved from phage biopanning
CRSHHSWSC

SEQ ID NO: 126 - protein, 9 aa, artificial sequence
  peptide with peptide binding sequence retrieved from phage biopanning
CKQFLSLSC

SEQ ID NO: 127 - protein, 9 aa, artificial sequence
  peptide with peptide binding sequence retrieved from phage biopanning
CDDASLRHC

SEQ ID NO: 128 - protein, 9 aa, artificial sequence
  peptide with peptide binding sequence retrieved from phage biopanning
CDNRGSQFC

SEQ ID NO: 129 - protein, 9 aa, artificial sequence
  peptide with peptide binding sequence retrieved from phage biopanning
CHHNLSSAC

SEQ ID NO: 130 - protein, 9 aa, artificial sequence
  peptide with peptide binding sequence retrieved from phage biopanning
CITGPTGAC

SEQ ID NO: 131 - protein, 9 aa, artificial sequence
  peptide with peptide binding sequence retrieved from phage biopanning
CPPGPTASC

SEQ ID NO: 132 - protein, 9 aa, artificial sequence
  peptide with peptide binding sequence retrieved from phage biopanning
CHQAGGHQC

SEQ ID NO: 133 - protein, 9 aa, artificial sequence
  peptide with peptide binding sequence retrieved from phage biopanning
CYFSWWHPC

SEQ ID NO: 134 - protein, 9 aa, artificial sequence
  peptide with peptide binding sequence retrieved from phage biopanning
CSPVKYPSC

SEQ ID NO: 135 - protein, 9 aa, artificial sequence
  peptide with peptide binding sequence retrieved from phage biopanning
CTSHFKLHC

SEQ ID NO: 136 - protein, 9 aa, artificial sequence
  peptide with peptide binding sequence retrieved from phage biopanning
CQQGTAPLC

SEQ ID NO: 137 - protein, 9 aa, artificial sequence
  peptide with peptide binding sequence retrieved from phage biopanning
CQEHSAKSC

SEQ ID NO: 138 - protein, 9 aa, artificial sequence
  peptide with peptide binding sequence retrieved from phage biopanning
CQTEDLPRC

SEQ ID NO: 139 - protein, 9 aa, artificial sequence
  peptide with peptide binding sequence retrieved from phage biopanning
CNRTSPAHC

SEQ ID NO: 140 - protein, 9 aa, artificial sequence
  peptide with peptide binding sequence retrieved from phage biopanning
CQGNHIGLC

SEQ ID NO: 141 - protein, 9 aa, artificial sequence
  peptide with peptide binding sequence retrieved from phage biopanning
CLNNYTHTC

SEQ ID NO: 142 - protein, 9 aa, artificial sequence
  peptide with peptide binding sequence retrieved from phage biopanning
CLTTASTKC

SEQ ID NO: 143 - protein, 9 aa, artificial sequence
  peptide with peptide binding sequence retrieved from phage biopanning
CLLSLRPAC

SEQ ID NO: 144 - protein, 9 aa, artificial sequence
  peptide with peptide binding sequence retrieved from phage biopanning
CDSQLWPIC

SEQ ID NO: 145 - protein, 9 aa, artificial sequence
  peptide with peptide binding sequence retrieved from phage biopanning
CDDRTTKIC

SEQ ID NO: 146 - protein, 9 aa, artificial sequence
  peptide with peptide binding sequence retrieved from phage biopanning
CWWPDGWYC

SEQ ID NO: 147 - protein, 9 aa, artificial sequence
  peptide with peptide binding sequence retrieved from phage biopanning
CKLQLTNQC

SEQ ID NO: 148 - protein, 9 aa, artificial sequence
  peptide with peptide binding sequence retrieved from phage biopanning
CWHGLGGNC

SEQ ID NO: 149 - protein, 9 aa, artificial sequence
  peptide with peptide binding sequence retrieved from phage biopanning
CHITLLKRC

SEQ ID NO: 150 - protein, 9 aa, artificial sequence
  peptide with peptide binding sequence retrieved from phage biopanning
CESMARPHC

SEQ ID NO: 151 - protein, 9 aa, artificial sequence
  peptide with peptide binding sequence retrieved from phage biopanning
CHWSWWHPC

SEQ ID NO: 152 - protein, 9 aa, artificial sequence
  peptide with peptide binding sequence retrieved from phage biopanning
CTLLLSRNC

SEQ ID NO: 153 - protein, 9 aa, artificial sequence
  peptide with peptide binding sequence retrieved from phage biopanning
CSSVSYMAC

SEQ ID NO: 154 - protein, 9 aa, artificial sequence
  peptide with peptide binding sequence retrieved from phage biopanning
CHWRWLPAC

SEQ ID NO: 155 - protein, 12 aa, artificial sequence
  peptide with peptide binding sequence retrieved from phage biopanning
WSPGQQRLHNSX

SEQ ID NO: 156 - protein, 12 aa, artificial sequence
  peptide with peptide binding sequence retrieved from phage biopanning
DSSNPIFWRPSS

SEQ ID NO: 157 - protein, 12 aa, artificial sequence
  peptide with peptide binding sequence retrieved from phage biopanning
EPFPASSLMTIR

SEQ ID NO: 158 - protein, 12 aa, artificial sequence
  peptide with peptide binding sequence retrieved from phage biopanning
SYHWDKTPQVLI

SEQ ID NO: 159 - protein, 12 aa, artificial sequence
  peptide with peptide binding sequence retrieved from phage biopanning
SGHQLLLNKMPN

SEQ ID NO: 160 - protein, 12 aa, artificial sequence
  peptide with peptide binding sequence retrieved from phage biopanning
SIPSEASLSSPR

SEQ ID NO: 161 - protein, 12 aa, artificial sequence
  peptide with peptide binding sequence retrieved from phage biopanning
TVPPQLNAQFRS

SEQ ID NO: 162 - protein, 12 aa, artificial sequence
  peptide with peptide binding sequence retrieved from phage biopanning
SDNVHTWQAMFK

SEQ ID NO: 163 - protein, 12 aa, artificial sequence
  peptide with peptide binding sequence retrieved from phage biopanning
YPSLLKMQPQFS

SEQ ID NO: 164 - protein, 12 aa, artificial sequence
  peptide with peptide binding sequence retrieved from phage biopanning
LPIPAHVAPHGP

SEQ ID NO: 165 - protein, 12 aa, artificial sequence
  peptide with peptide binding sequence retrieved from phage biopanning
LWGRPFPDLLHQ

SEQ ID NO: 166 - protein, 12 aa, artificial sequence
  peptide with peptide binding sequence retrieved from phage biopanning
QTPPWILSHPPQ

SEQ ID NO: 167 - protein, 12 aa, artificial sequence
  peptide with peptide binding sequence retrieved from phage biopanning
NHPHPTPARGII

SEQ ID NO: 168 - protein, 12 aa, artificial sequence
  peptide with peptide binding sequence retrieved from phage biopanning
HPSSAPWGVALA

SEQ ID NO: 169 - protein, 11 aa, artificial sequence
  peptide with peptide binding sequence retrieved from phage biopanning
HWNHRYSMWGA

SEQ ID NO: 170 - protein, 12 aa, artificial sequence
  peptide with peptide binding sequence retrieved from phage biopanning
NHRIWESFWPSA

SEQ ID NO: 171 - protein, 12 aa, artificial sequence
  peptide with peptide binding sequence retrieved from phage biopanning
HSSWWLALAKPT

SEQ ID NO: 172 - protein, 12 aa, artificial sequence
  peptide with peptide binding sequence retrieved from phage biopanning
SNNDLSPLQTSH

SEQ ID NO: 173 - protein, 12 aa, artificial sequence
  peptide with peptide binding sequence retrieved from phage biopanning
SGLPHLSLNAPR

SEQ ID NO: 174 - protein, 12 aa, artificial sequence
  peptide with peptide binding sequence retrieved from phage biopanning
SWPLYSRDSGLG

SEQ ID NO: 175 - protein, 12 aa, artificial sequence
  peptide with peptide binding sequence retrieved from phage biopanning
LPGWPLAERVGQ

SEQ ID NO: 176 - protein, 12 aa, artificial sequence
  peptide with peptide binding sequence retrieved from phage biopanning
SHPWNAQRELSV

SEQ ID NO: 177 - protein, 12 aa, artificial sequence
  peptide with peptide binding sequence retrieved from phage biopanning
VSRHQSWHPHDL

SEQ ID NO: 178 - protein, 12 aa, artificial sequence
  peptide with peptide binding sequence retrieved from phage biopanning
YWPSKHWWWLAP

SEQ ID NO: 179 - protein, 12 aa, artificial sequence
  peptide with peptide binding sequence retrieved from phage biopanning
SSAWWSYWPPVA

SEQ ID NO: 180 - protein, 12 aa, artificial sequence
  peptide with peptide binding sequence retrieved from phage biopanning
APLGFNSMRLPA

SEQ ID NO: 181 - protein, 12 aa, artificial sequence
  peptide with peptide binding sequence retrieved from phage biopanning
WNMRWLPTWAPA

SEQ ID NO: 182 - protein, 12 aa, artificial sequence
  peptide with peptide binding sequence retrieved from phage biopanning
WPRYPSTLVSSH

SEQ ID NO: 183 - protein, 12 aa, artificial sequence
  peptide with peptide binding sequence retrieved from phage biopanning
GKESVPPPRIYA

SEQ ID NO: 184 - protein, 12 aa, artificial sequence
  peptide with peptide binding sequence retrieved from phage biopanning
LTLDMKRTSGPL

SEQ ID NO: 185 - protein, 12 aa, artificial sequence
  peptide with peptide binding sequence retrieved from phage biopanning
LSTHTTESRSMV

SEQ ID NO: 186 - protein, 12 aa, artificial sequence
  peptide with peptide binding sequence retrieved from phage biopanning
EYLSAIVAGPWP

SEQ ID NO: 187 - protein, 12 aa, artificial sequence
  peptide with peptide binding sequence retrieved from phage biopanning
QFKWWHSLSPTP

SEQ ID NO: 188 - protein, 12 aa, artificial sequence
  peptide with peptide binding sequence retrieved from phage biopanning
APTPLIGKRLVQ

SEQ ID NO: 189 - protein, 12 aa, artificial sequence
  peptide with peptide binding sequence retrieved from phage biopanning
LINPRDHVLAPQ

SEQ ID NO: 190 - protein, 12 aa, artificial sequence
  peptide with peptide binding sequence retrieved from phage biopanning
LLADTTHHRPWT

SEQ ID NO: 191 - protein, 12 aa, artificial sequence
  peptide with peptide binding sequence retrieved from phage biopanning
QASISPLWTPTP

SEQ ID NO: 192 - protein, 12 aa, artificial sequence
  peptide with peptide binding sequence retrieved from phage biopanning
NSXLHLAHQPHK

SEQ ID NO: 193 - protein, 12 aa, artificial sequence
  peptide with peptide binding sequence retrieved from phage biopanning
TKNMLSLPVGPG

SEQ ID NO: 194 - protein, 12 aa, artificial sequence
  peptide with peptide binding sequence retrieved from phage biopanning
DMPRTTMSPPPR

SEQ ID NO: 195 - protein, 12 aa, artificial sequence
  peptide with peptide binding sequence retrieved from phage biopanning
STPALMTLIART

SEQ ID NO: 196 - protein, 12 aa, artificial sequence
  peptide with peptide binding sequence retrieved from phage biopanning
TSNFINRMNPGL

SEQ ID NO: 197 - protein, 12 aa, artificial sequence
  peptide with peptide binding sequence retrieved from phage biopanning
TSASTRPELHYP

SEQ ID NO: 198 - protein, 12 aa, artificial sequence
  peptide with peptide binding sequence retrieved from phage biopanning
NLLEVISLPHRG

SEQ ID NO: 199 - protein, 12 aa, artificial sequence
  peptide with peptide binding sequence retrieved from phage biopanning
QHPNNAHVRQFP

SEQ ID NO: 200 - protein, 12 aa, artificial sequence
  peptide with peptide binding sequence retrieved from phage biopanning
QHANNQAWNNLR

SEQ ID NO: 201 - protein, 12 aa, artificial sequence
  peptide with peptide binding sequence retrieved from phage biopanning
QHYPGRAIPHST

SEQ ID NO: 202 - protein, 12 aa, artificial sequence
  peptide with peptide binding sequence retrieved from phage biopanning
VPPPHPQFDHLI

SEQ ID NO: 203 - protein, 12 aa, artificial sequence
  peptide with peptide binding sequence retrieved from phage biopanning
LKMNPSISSSLK

SEQ ID NO: 204 - protein, 12 aa, artificial sequence
  peptide with peptide binding sequence retrieved from phage biopanning
HWDPFSLSAYFP

SEQ ID NO: 205 - protein, 12 aa, artificial sequence
  peptide with peptide binding sequence retrieved from phage biopanning
WSPGQQRLHNST

SEQ ID NO: 206 - protein, 12 aa, artificial sequence
  peptide with peptide binding sequence retrieved from phage biopanning
NMTKHPLAYTEP

SEQ ID NO: 207 - protein, 12 aa, artificial sequence
  peptide with peptide binding sequence retrieved from phage biopanning
HMPTKSASQTYF

SEQ ID NO: 208 - protein, 12 aa, artificial sequence
  peptide with peptide binding sequence retrieved from phage biopanning
HNAYWHWPPSMT

SEQ ID NO: 209 - protein, 12 aa, artificial sequence
  peptide with peptide binding sequence retrieved from phage biopanning
VLPPKPMRQPVA

SEQ ID NO: 210 - protein, 12 aa, artificial sequence
  peptide with peptide binding sequence retrieved from phage biopanning
SLHKISQLSFAS

SEQ ID NO: 211 - protein, 12 aa, artificial sequence
  peptide with peptide binding sequence retrieved from phage biopanning
WHSRLPPMTVAF

SEQ ID NO: 212 - protein, 12 aa, artificial sequence
  peptide with peptide binding sequence retrieved from phage biopanning
TPWFQWHQWNLN

SEQ ID NO: 213 - protein, 12 aa, artificial sequence
  peptide with peptide binding sequence retrieved from phage biopanning
SDTISRLHVSMT

SEQ ID NO: 214 - protein, 12 aa, artificial sequence
  peptide with peptide binding sequence retrieved from phage biopanning
NPYHPTIPQSVH

SEQ ID NO: 215 - protein, 12 aa, artificial sequence
  peptide with peptide binding sequence retrieved from phage biopanning
LPSAKLPPGPPK

SEQ ID NO: 216 - protein, 12 aa, artificial sequence
  peptide with peptide binding sequence retrieved from phage biopanning
TSNPHTRHYYPI

SEQ ID NO: 217 - protein, 12 aa, artificial sequence
  peptide with peptide binding sequence retrieved from phage biopanning
SNFTTQMTFYTG

SEQ ID NO: 218 - protein, 12 aa, artificial sequence
  peptide with peptide binding sequence retrieved from phage biopanning
KMDRHDPSPALL

SEQ ID NO: 219 - protein, 12 aa, artificial sequence
  peptide with peptide binding sequence retrieved from phage biopanning
MPAVMSSAQVPR

SEQ ID NO: 220 - protein, 12 aa, artificial sequence
  peptide with peptide binding sequence retrieved from phage biopanning
DRAPLIPFASQH

SEQ ID NO: 221 - protein, 12 aa, artificial sequence
  peptide with peptide binding sequence retrieved from phage biopanning
DQYIQQAHRSHI

SEQ ID NO: 222 - protein, 12 aa, artificial sequence
  peptide with peptide binding sequence retrieved from phage biopanning
HARINPSFPLPI

SEQ ID NO: 223 - protein, 12 aa, artificial sequence
  peptide with peptide binding sequence retrieved from phage biopanning
GWWPYAALRALS

SEQ ID NO: 224 - protein, 12 aa, artificial sequence
  peptide with peptide binding sequence retrieved from phage biopanning
TAATSSPHSRSP

SEQ ID NO: 225 - protein, 12 aa, artificial sequence
  peptide with peptide binding sequence retrieved from phage biopanning
STTGQSPALAPP

SEQ ID NO: 226 - protein, 12 aa, artificial sequence
  peptide with peptide binding sequence retrieved from phage biopanning
HSSWYIQHFPPL

SEQ ID NO: 227 - protein, 12 aa, artificial sequence
  peptide with peptide binding sequence retrieved from phage biopanning
GSHSNPTPLTPR

SEQ ID NO: 228 - protein, 12 aa, artificial sequence
  peptide with peptide binding sequence retrieved from phage biopanning
YTGVLDTKATQN

SEQ ID NO: 229 - protein, 7 aa, artificial sequence
  peptide with peptide binding sequence retrieved from phage biopanning
NNPHMQN

SEQ ID NO: 230 - protein, 12 aa, artificial sequence
  peptide with peptide binding sequence retrieved from phage biopanning
VISNHAESSRRL

SEQ ID NO: 231 - protein, 7 aa, artificial sequence
  peptide with peptide binding sequence retrieved from phage biopanning
DSPHRHS

SEQ ID NO: 232 - protein, 8 aa, artificial sequence
  peptide with peptide binding sequence retrieved from phage biopanning
NNPMHQNC

SEQ ID NO: 233 - protein, 10 aa, artificial sequence
  peptide with peptide binding sequence retrieved from phage biopanning
SGPAHGMFAR

SEQ ID NO: 234 - protein, 9 aa, artificial sequence
  peptide with peptide binding sequence retrieved from phage biopanning
CTYSRLHLC

SEQ ID NO: 235 - protein, 9 aa, artificial sequence
  peptide with peptide binding sequence retrieved from phage biopanning
CRPYNIHQC

SEQ ID NO: 236 - protein, 9 aa, artificial sequence
  peptide with peptide binding sequence retrieved from phage biopanning
CPFKTAFPC

SEQ ID NO: 237 - protein, 7 aa, artificial sequence
  peptide with peptide binding sequence retrieved from phage biopanning
XXPMHXX

SEQ ID NO: 238 - protein, 7 aa, artificial sequence
  peptide with peptide binding sequence retrieved from phage biopanning
WWSWHPW

SEQ ID NO: 239 - protein, 7 aa, artificial sequence
  peptide with peptide binding sequence retrieved from phage biopanning
HWSWWHP

SEQ ID NO: 240 - protein, 14 aa, artificial sequence
  peptide with peptide binding sequence retrieved from phage biopanning
ACWWSWHPWCGGGK

SEQ ID NO: 241 - protein, 14 aa, artificial sequence
  peptide with peptide binding sequence retrieved from phage biopanning
ACDSPHRHSCGGGK

SEQ ID NO: 242 - protein, 14 aa, artificial sequence
  peptide with peptide binding sequence retrieved from phage biopanning
ACPRSSHDHCGGGK

SEQ ID NO: 243 - protein, 7 aa, artificial sequence
  peptide with peptide binding sequence retrieved from phage biopanning
YFSWWHP

SEQ ID NO: 244 - protein, 16 aa, artificial sequence
  peptide with peptide binding sequence retrieved from phage biopanning
DMPRTTMSPPPRGGGK

SEQ ID NO: 245 - protein, 12 aa, artificial sequence
  peptide with peptide binding sequence retrieved from phage biopanning
NHRIWESFWPSA